(19) United States
(12) Patent Application Publication
(10) Pub. No.: US 2024/0024644 A1
(43) Pub. Date: Jan. 25, 2024

(54) TISSUE PENETRATING AND BIODEGRADABLE DRUG DELIVERY DEVICES AND METHODS OF USE THEREOF

(71) Applicant: PanTher Therapeutics, Inc., Watertown, MA (US)

(72) Inventors: Laura INDOLFI, Boston, MA (US); Margaret LASHOF-SULLIVAN, Watertown, MA (US); Daniel WILDMAN, Wellington, FL (US); Christopher D. L. JOHNSON, Acton, MA (US)

(73) Assignee: PanTher Therapeutics, Inc., Watertown, MA (US)

(21) Appl. No.: 18/356,925

(22) Filed: Jul. 21, 2023

Related U.S. Application Data
(60) Provisional application No. 63/391,535, filed on Jul. 22, 2022.
(60) Provisional application No. 63/501,986, filed on May 12, 2023.

(51) Int. Cl. A61M 31/00; A61B 34/30; A61M 37/00; A61K 47/59

(52) U.S. Cl. CPC A61M 31/002; A61B 34/30; A61M 37/00; A61K 47/593

(57) ABSTRACT

Provided are tissue penetrating and biodegradable drug delivery devices capable of delivering an active pharmaceutical ingredient (API) directly to a target tissue site. The drug delivery devices can include a plurality of layers, wherein at least one of the layers of the plurality of layers includes an API and a biodegradable polymer. When the device is placed directly into or adjacent to the target tissue, the API layer can degrade thereby releasing the API multi-directionally into the target tissue.

### CROSS-REFERENCE TO RELATED APPLICATIONS

This application claims the benefit of U.S. Provisional Application No. 63/391,535 filed Jul. 22, 2022, and U.S. Provisional Application No. 63/501,986 filed May 12, 2023, the entire contents of each of which are incorporated herein by reference.

### FIELD OF THE DISCLOSURE

This relates to implantable drug delivery devices. Specifically, this relates to tissue penetrating and biodegradable drug delivery devices that deliver active pharmaceutical ingredients to local tissue sites.

### BACKGROUND OF THE DISCLOSURE

Cancer therapies have advanced significantly in recent years. Challenges remain, however, including limited ability of drugs to successfully reach the tumor, short half-life, and low retention rate on site. A major limit of existing chemotherapy effectiveness is due to systemic off-target toxicity. For example, only 1-5% of a chemotherapy dose via systemic administration actually reaches the tumor site. In fact, even some of the most promising currently available chemotherapies are not tolerable in the long term for most patients.

### SUMMARY OF THE DISCLOSURE

Applicant has discovered an implantable, tissue penetrating, and biodegradable drug delivery device capable of delivering an active pharmaceutical ingredient (API) directly to the target tissue in the patient. Since the drug delivery devices disclosed herein utilize a local delivery method, these devices can sidestep the problem of systemic side effects and can more directly provide site-specific treatment without having to perform subsequent removal surgeries due to their biodegradable nature. Specifically, the drug devices disclosed herein can be flexibly applied via open and minimally invasive surgery, allow for unidirectional, multidirectional, or omni-directional delivery of APIs via a multilayer configuration, act as a barrier to tumor ingrowth and prevent systemic drug leakage, provide tunable release profiles and degradation rates, be combinable with systemic therapy, and/or consent adaptable design translatable to a broad range of solid tumors.

In some embodiments, the drug delivery devices can be flexible. In some embodiments, the drug delivery devices can be flexible such that they can be placed into (i.e., intratumorally) and/or adjacent to target tissue in a patient using standard open, laparoscopic, endoscopic (e.g., bronchoscopic), percutaneous, or robotic surgical equipment. In some embodiments, the drug delivery devices can be rigid. In some embodiments, without being flexible, the drug delivery devices may not be able to go through the tortuous route to the target tissue using a laparoscope, endoscope, bronchoscope, etc. In some embodiments, the drug delivery device can be partially or fully embedded into a target tissue site. In some embodiments, multiple drug delivery devices can be placed into and/or adjacent to the target tissue (i.e., intratumorally) in a patient using standard open, laparoscopic, percutaneous, endoscopic, or robotic surgical equipment. In some embodiments, a second drug delivery device can be placed into and/or adjacent to the target tissue of a patient after the first drug delivery device has already degraded or simultaneously with the first drug delivery device.

In some embodiments, the drug delivery devices disclosed herein can include at least two layers. In some embodiments, at least one of the layers can include an API and a biodegradable polymer. In some embodiments, at least one of the layers can include a different API and a biodegradable polymer. The biodegradable polymer with the different API can degrade slower, faster, or the same as the biodegradable polymer of the at least one layer with the first API. In some embodiments, at least one of the layers includes a biodegradable polymer without an API. In some embodiments, the non-API layer can be a support layer for providing protection for the API layers, structural support to the drug delivery device, and/or strength to the drug delivery device. In some embodiments, all layers of the drug delivery device can include an API.

When the device is placed directly in a target tissue, the API layer(s) can degrade thereby releasing the API(s) in the target tissue. In some embodiments, the non-API layer or a non-API component(s) (e.g., the tip as discussed herein) of the drug delivery device can prevent the drug delivery device from being released from the target tissue (i.e., the non-API layer or non-API component can hold the drug delivery device in place). In some embodiments, an API layer or layers can prevent the drug delivery device from being released from the target tissue.

In some embodiments, a non-API layer (or another non-API component like the tip) can degrade more slowly than at least one of the API layers such that the non-API layer can continue to provide support for the drug delivery device and/or retain the drug delivery device in the target tissue site. As such, the non-API layer(s) or non-API component may not degrade completely until the drug is completely released. Accordingly, the drug delivery device can be inserted in a patient to target specific tissue without having to perform a subsequent surgery to remove the device as it can be wholly or mostly absorbed by the patient's body.

The drug delivery devices disclosed herein can provide clinically relevant delivery of an API directly to, in, or adjacent to tissue (e.g., a tumor) with good tolerance and minimal systemic exposure of the API. This device can be used as a neoadjuvant therapy (and/or adjuvant therapy) for treatment of many internal medical issues (e.g., cancer, other diseases, wounds, sores, lacerations, non-cancerous growths, etc.) that may improve complete resection rates, reduce the risk of local recurrence post-resection, reduce and/or control tumor mass, and/or improve survival of patients, along with all the benefits associated with the above.

In some embodiments, a drug delivery device includes a body comprising a plurality of layers, wherein the plurality of layers comprises at least one first layer comprising an active pharmaceutical ingredient (API) and a biodegradable polymer, wherein the drug delivery device is configured to be inserted into a target tissue site of a patient. In some embodiments, the device includes a tip connected or integrated to a distal end of the body of the drug delivery device. In some embodiments, the drug delivery device is configured to be inserted into a target tissue site of the patient tip first. In some embodiments, the target tissue site is a tumor of the pancreas, biliary system, gallbladder, esophageal system, liver, stomach, peritoneum, small bowel, lung, or colon. In some embodiments, the biodegradable polymer is poly(lactic-co-glycolic acid) (PLGA). In some embodiments, the biodegradable polymer is PLGA 50:50. In some embodiments, the at least one first layer comprises 1-15 wt. % API. In some embodiments, the at least one first layer comprises a solvent. In some embodiments, the at least one first layer comprises 1-15 wt. % solvent. In some embodiments, the plurality of layers comprises at least one second layer comprising a second biodegradable polymer, wherein the second biodegradable polymer has a slower degradation rate than the first biodegradable polymer. In some embodiments, the second biodegradable polymer is PLGA 75:25. In some embodiments, the plurality of layers comprises at least one third layer comprising a second API and a third biodegradable polymer. In some embodiments, the plurality of layers comprises at least one fourth layer comprising a third API and a fourth biodegradable polymer. In some embodiments, each of the plurality of layers has the same composition. In some embodiments, the drug delivery device is configured to be implanted in a patient using standard open and/or minimally invasive procedures. In some embodiments, the drug delivery device is configured to be inserted into a target tissue site of a patient using a bronchoscope, forceps, trocar, or needle. In some embodiments, the drug delivery device is configured to be inserted into a target tissue site of a patient robotically. In some embodiments, the at least one first layer is configured to release the API when inserted into a target tissue site of a patient. In some embodiments, the at least one first layer is configured to release the API multi-directionally when inserted into the target tissue site of the patient. In some embodiments, the release of the API is controlled by in vivo degradation of the biodegradable polymer in the target tissue site. In some embodiments, the tip comprises a fifth biodegradable polymer that has a slower degradation rate than the first biodegradable polymer. In some embodiments, the tip comprises PLGA 75:25. In some embodiments, the tip comprises a tissue retaining mechanism. In some embodiments, the tissue retaining mechanism is configured to hold the drug delivery device in a target tissue when inserted in the target tissue of a patient. In some embodiments, the tissue retaining mechanism extends outwardly from the tip. In some embodiments, the tissue retaining mechanism comprises barbs and/or edges. In some embodiments, the tip has at least one vertex at its distal end. In some embodiments, the body comprises a disengagement mechanism towards a proximal end of the body. In some embodiments, a flexible shaft is connected to the proximal end of the body. In some embodiments, the disengagement mechanism is configured to disengage the drug delivery device from the flexible shaft. In some embodiments, the disengagement mechanism is configured to disengage the drug delivery device from the flexible shaft via twisting. In some embodiments, the body of the drug delivery device is flexible.

In some embodiments, a method of preparing a drug delivery device includes adding a first solution comprising a first biodegradable polymer, an active pharmaceutical ingredient (API), and a solvent to a first substrate; drying the first solution on the first substrate to form a first layer; adding a second solution comprising a second biodegradable polymer and a second solvent to a second substrate; drying the second solution on the second substrate to form a second layer; laminating the first layer and the second layer together to form a laminated sheet. In some embodiments, the method includes cutting the laminated sheet to form a body of a drug delivery device. In some embodiments, the cutting is die cutting. In some embodiments, the second solution comprises a second API. In some embodiments, the first API and the second API are the same. In some embodiments, the method includes adding a third solution comprising a third biodegradable polymer and a third solvent a third substrate; drying the third solution on the third substrate to form a third layer; laminating the third layer to the laminated sheet to form a second laminated sheet. In some embodiments, spreading the first solution across the first substrate with a film applicator; and spreading the second solution across the second substrate with a film applicator. In some embodiments, the spread first solution and the spread second solution have a desired thickness pursuant to the film applicator. In some embodiments, the first and/or second substrate comprises a release liner. In some embodiments, the method includes heating the first and second layers in an oven after drying the first and second solutions. In some embodiments, the method includes attaching a tip to a distal end of the body of the drug delivery device.

In some embodiments, a method of treating tissue of a patient includes implanting a drug delivery device into a target tissue site of a patient, the drug delivery device comprising: a body comprising a plurality of layers, wherein the plurality of layers comprises at least one first layer comprising an active pharmaceutical ingredient (API) and a biodegradable polymer; and releasing the API from the at least one first layer in the target tissue site, wherein the release of the API is controlled by in vivo degradation of the biodegradable polymer in the target tissue site. In some embodiments, the drug delivery device comprises a tip connected to a distal end of the drug delivery device. In some embodiments, the drug delivery device is implanted tip first into the target tissue site of the patient. In some embodiments, the target tissue site is a tumor of the pancreas, biliary system, gallbladder, esophageal system, liver, stomach, peritoneum, small bowel, lung, or colon. In some embodiments, the drug delivery device is implanted via open surgery, laparoscopically, percutaneously, robotically, or endoscopically. In some embodiments, the drug delivery device is implanted via a bronchoscope. In some embodiments, releasing the API from the at least one first layer comprises releasing the API multi-directionally away from drug delivery device. In some embodiments, the drug delivery device is implanted into the target tissue site of the patient such that the tip is configured to hold the drug delivery device in the target tissue. In some embodiments, implanting the drug delivery device comprises using a tool to deliver the drug delivery device to the target tissue site of the patient. In some embodiments, the tool comprises a flexible shaft and is connected to a proximal end of the body of the drug delivery device. In some embodiments, implanting the drug delivery device into the target tissue site comprises disengaging the body of the drug delivery device from the flexible shaft. In some embodiments, disengaging the drug delivery device from the flexible shaft comprises twisting the flexible shaft with respect to the drug delivery device. In some embodiments, the body of the drug delivery device comprises an anatomical marker and implanting the drug delivery device into the target tissue site of the patient comprises monitoring a location the anatomical marker. In some embodiments, the release of the API follows a delay period after implantation. In some embodiments, a sub-therapeutically effective amount of the API is released during the delay period. In some embodiments, after the delay period, the API is released at a substantially linear or linear release rate.

In some embodiments, a drug delivery device includes a body comprising: a core comprising a first biodegradable polymer; and an outer layer surrounding at least a portion of the core comprising an active pharmaceutical ingredient (API) and a second biodegradable polymer, wherein the first biodegradable polymer has a slower degradation rate than the second biodegradable polymer; and a tip connected or integrated to a distal end of the body. In some embodiments, the drug delivery device is configured to be inserted into a target tissue site of a patient. In some embodiments, the drug delivery device is configured to be inserted into a target tissue site of the patient tip first. In some embodiments, the target tissue site is a tumor of the pancreas, biliary system, gallbladder, esophageal system, liver, stomach, peritoneum, small bowel, lung, or colon. In some embodiments, the first biodegradable polymer is poly(lactic-co-glycolic acid) (PLGA). In some embodiments, the first biodegradable polymer is PLGA 75:25. In some embodiments, the second biodegradable polymer is poly(lactic-co-glycolic acid) (PLGA). In some embodiments, the second biodegradable polymer is PLGA 50:50. In some embodiments, the drug delivery device is configured to be implanted in a patient using standard open and minimally invasive procedures. In some embodiments, the drug delivery device is configured to be inserted into a target tissue site of a patient using a bronchoscope. In some embodiments, the drug delivery device is configured to be inserted into a target tissue site of a patient robotically. In some embodiments, the outer layer is configured to release the API when inserted into a target tissue site of a patient. In some embodiments, the outer layer is configured to release the API omnidirectionally when inserted into the target tissue site of the patient. In some embodiments, the release of the API is controlled by in vivo degradation of the second biodegradable polymer in the target tissue site. In some embodiments, the body further comprises a second outer layer surrounding at least a portion of the first outer layer and comprising a third biodegradable polymer, wherein the third biodegradable polymer has a faster degradation rate than the second and/or first biodegradable polymer. In some embodiments, the third biodegradable polymer is PLGA. In some embodiments, the tip comprises a third biodegradable polymer that has a slower degradation rate than the second biodegradable polymer. In some embodiments, the tip comprises PLGA 75:25. In some embodiments, the tip comprises a tissue retaining mechanism. In some embodiments, the tissue retaining mechanism is configured to hold the drug delivery device in a target tissue when inserted in the target tissue of a patient. In some embodiments, the tissue retaining mechanism extends outwardly from the tip. In some embodiments, the tissue retaining mechanism comprises barbs and/or edges. In some embodiments, the tip has at least one vertex at its distal end. In some embodiments, the body comprises a disengagement mechanism towards a proximal end of the body. In some embodiments, a flexible shaft is connected to the proximal end of the body. In some embodiments, the disengagement mechanism is configured to disengage the drug delivery device from the flexible shaft. In some embodiments, the disengagement mechanism is configured to disengage the drug delivery device from the flexible shaft via twisting.

In some embodiments, a drug delivery device includes a core comprising a first biodegradable polymer; and an outer layer surrounding at least a portion of the core comprising an active pharmaceutical ingredient (API) and a second biodegradable polymer, wherein the first biodegradable polymer has a slower degradation rate than the second biodegradable polymer. In some embodiments, the drug delivery device is configured to be inserted into a target tissue site of a patient. In some embodiments, the target tissue site is a solid tumor of the pancreas, biliary system, gallbladder, esophageal system, liver, stomach, peritoneum, small bowel, lung, or colon. In some embodiments, the first biodegradable polymer is poly(lactic-co-glycolic acid) (PLGA). In some embodiments, the first biodegradable polymer is PLGA 75:25. In some embodiments, the second biodegradable polymer is poly(lactic-co-glycolic acid) (PLGA). In some embodiments, the second biodegradable polymer is PLGA 50:50. In some embodiments, the drug delivery device is configured to be implanted in a patient using standard open and minimally invasive procedures. In some embodiments, the drug delivery device is configured to be inserted into a target tissue site of a patient using a bronchoscope. In some embodiments, the drug delivery device is configured to be inserted into a target tissue site of a patient robotically. In some embodiments, the outer layer is configured to release the API when inserted into a target tissue site of a patient. In some embodiments, the outer layer is configured to release the API omnidirectionally when inserted into the target tissue site of the patient. In some embodiments, the release of the API is controlled by in vivo degradation of the second biodegradable polymer in the target tissue site.

In some embodiments, a method of treating tissue of a patient includes implanting a drug delivery device into a target tissue site of a patient, the drug delivery device comprising: a body comprising: a core comprising a first biodegradable polymer; and an outer layer surrounding at least a portion of the core comprising an active pharmaceutical ingredient (API) and a second biodegradable polymer, wherein the first biodegradable polymer has a slower degradation rate than the second biodegradable polymer; and a tip connected to a distal end of the body; and releasing the API from the outer layer in the target tissue site, wherein the release of the API is controlled by in vivo degradation of the second biodegradable polymer in the target tissue site. In some embodiments, the drug delivery device is implanted tip first into the target tissue site of the patient. In some embodiments, the target tissue site is a tumor of the pancreas, biliary system, gallbladder, esophageal system, liver, stomach, peritoneum, small bowel, lung, or colon. In some embodiments, the drug delivery device is implanted via open surgery, laparoscopically, robotically, percutaneously, or endoscopically. In some embodiments, the drug delivery device is implanted via a bronchoscope. In some embodiments, releasing the API from the outer layer comprises releasing the API omnidirectionally away from the outer layer. In some embodiments, the drug delivery device is implanted into the target tissue site of the patient such that the tip is configured to hold the drug delivery device in the target tissue. In some embodiments, implanting the drug delivery device comprises using a tool to deliver the drug delivery device to the target tissue site of the patient. In some embodiments, the tool comprises a flexible shaft and is connected to the proximal end of the body. In some embodiments, implanting the drug delivery device into the target tissue site comprises disengaging the body of the drug delivery device from the flexible shaft. In some embodiments, disengaging the drug delivery device from the flexible shaft comprises twisting the flexible shaft with respect to the drug delivery device.

Additional advantages will be readily apparent to those skilled in the art from the following detailed description. The examples and descriptions herein are to be regarded as illustrative in nature and not restrictive.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### BRIEF DESCRIPTION OF THE DRAWINGS

The invention will now be described, by way of example only, with reference to the accompanying drawings.

FIG. 1A illustrates a top view of a drug delivery device in accordance with some embodiments disclosed herein.

FIG. 1B illustrates a side view of the drug delivery device in accordance with some embodiments disclosed herein.

FIG. 1C illustrates a cross section along line C-C of FIG. 1B in accordance with some embodiments disclosed herein.

FIG. 1D illustrates another rectangular prism-shaped drug delivery device in accordance with some embodiments disclosed herein.

FIG. 2A illustrates another drug delivery device in accordance with some embodiments disclosed herein.

FIG. 2B illustrates a cross section along line A-A of FIG. 2A in accordance with some embodiments disclosed herein.

FIG. 2C illustrates a cross section along line B-B of FIG. 2A in accordance with some embodiments disclosed herein.

FIG. 2D illustrates forceps connected to a drug delivery device in accordance with some embodiments disclosed herein.

FIG. 2E illustrates a cross section of a drug delivery device having a hollow core in accordance with some embodiments disclosed herein.

FIG. 2F illustrates another cross section of a drug delivery device having a hollow core in accordance with some embodiments disclosed herein.

FIG. 3 illustrates applying a biodegradable polymer/API solution to a substrate using a film applicator in accordance with some embodiments disclosed herein.

FIG. 4A illustrates images of drug delivery devices in accordance with some embodiments disclosed herein.

FIG. 4B illustrates additional images of drug delivery devices in accordance with some embodiments disclosed herein.

FIG. 4C illustrates two additional images of various sizes of drug delivery devices in accordance with some embodiments disclosed herein.

FIG. 5 illustrates the cumulative drug release of a drug delivery device tested in accordance with some embodiments disclosed herein.

In the Figures, like reference numbers correspond to like components unless otherwise stated.

### DETAILED DESCRIPTION OF THE DISCLOSURE

Described herein are exemplary embodiments of biodegradable drug delivery devices that can be implanted in a patient to locally deliver a controlled therapeutically effective amount of an API. Specifically, the drug delivery devices disclosed herein can be configured to provide controlled release of a therapeutically effective amount of an API directly to or into and/or adjacent to a target tissue site (e.g., a tumor) by in vivo degradation of a biodegradable polymer layer containing the API. In some embodiments, it may not be possible to place the drug delivery device directly into a target tissue site (e.g., a tumor). As such, the drug delivery device can be delivered adjacent to the target tissue. For example, a tumor may be encased in a scar capsule and/or it may be indicated to not be “poked” to avoid disrupting the tumor as it might expel unwanted components. In these situations, the drug delivery device can be placed adjacent to the tumor site such as close to the edge or border of the tumor site and/or touching or in contact with the tumor site.

In some embodiments, the targeted tissue can be cancerous tissue/cells or peritumoral tissue/cells on an organ. For example, the targeted tissue can be cancerous or peritumoral tissue/cells on a pancreas, biliary system, gallbladder, esophageal system, liver, stomach, peritoneum, small bowel, lung, colon, or metastasis from a primary tumor.

PCT Application No. PCT/US2022/079996 (which is hereby incorporated by reference in its entirety) discloses a film or patch drug delivery device having multiple biodegradable layers. In use, the API layer disclosed in PCT/US2022/079996 would face the targeted tissue such that the API is released toward the targeted tissue during degradation and the non-API backing layer can prevent API from being released away from the targeted tissue onto non-targeted tissue.

As disclosed herein, in some embodiments, the drug delivery device can be a tissue (e.g., tumor) penetrating device (like a bullet, arrow, plug, or other such device) having multiple biodegradable layers (e.g., core layers, inner layers, shell layers, and/or outer layers). At least one layer or multiple layers of the device can include an API which can be released in vivo by polymer biodegradation. In some embodiments, at least one layer can be free of any API. In some embodiments, no layer may be free of any API (i.e., all layers include at least some API). In some embodiments, a non-API layer(s) can be a core or core layer or inner layer of the drug delivery device. In some embodiments, a non-API layer(s) can be an outer layer or shell layer of the drug delivery device.

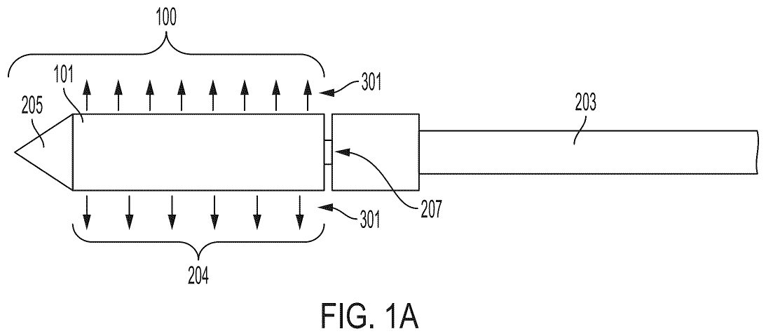

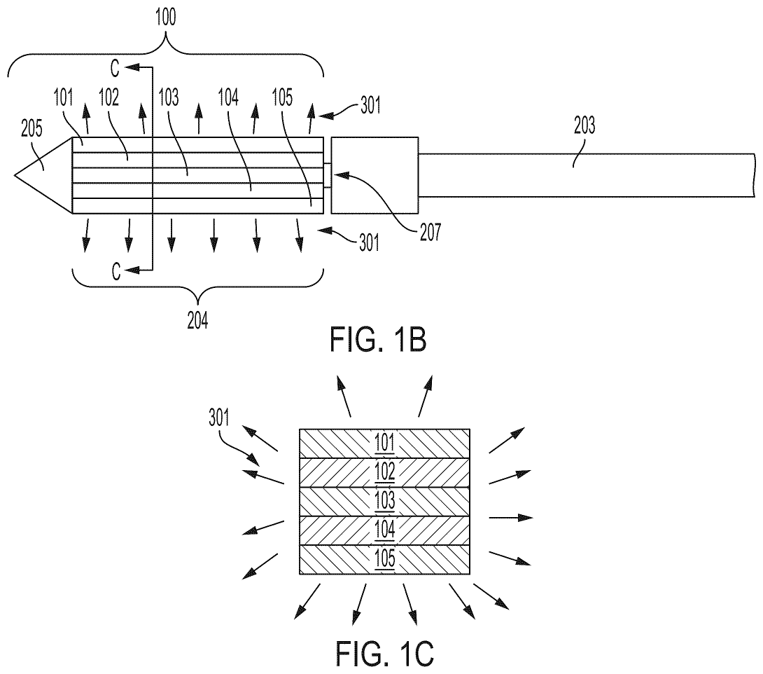

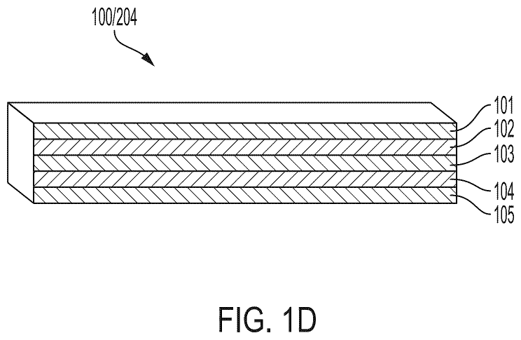

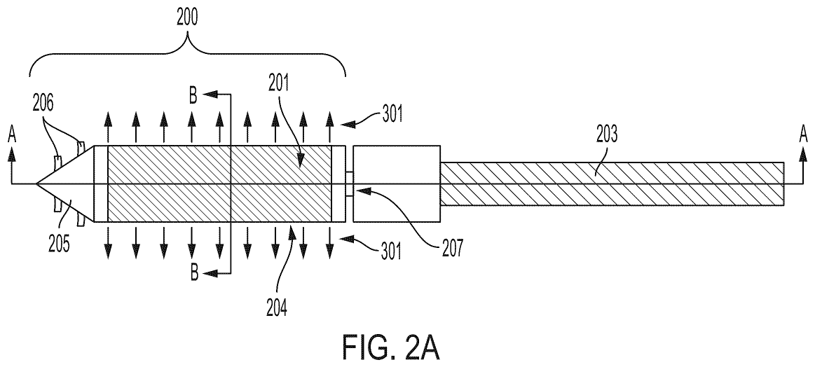

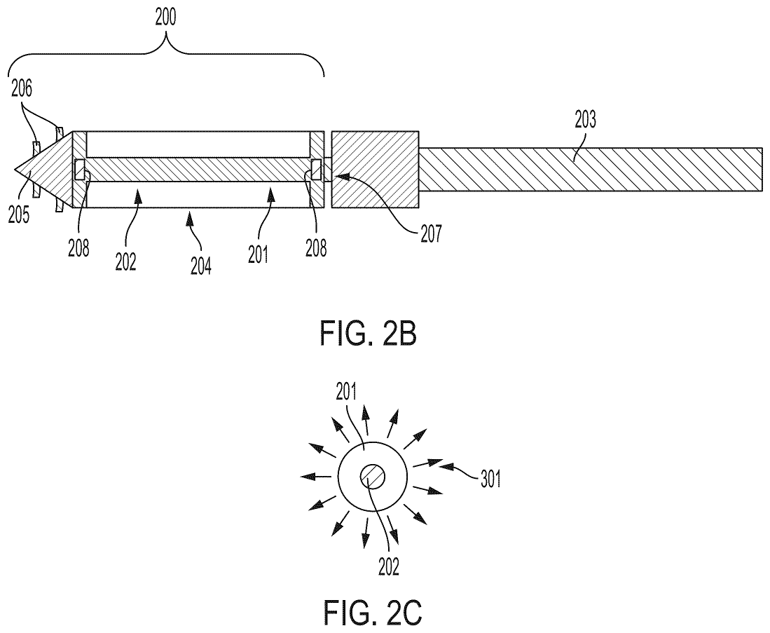

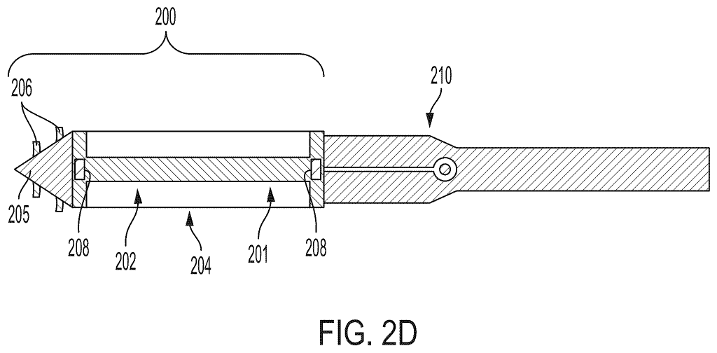

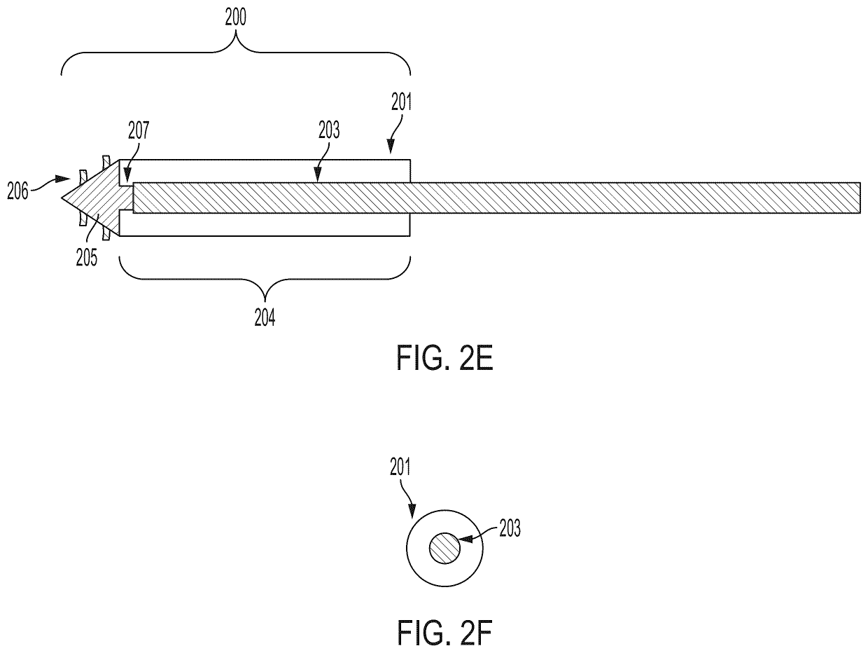

The drug delivery devices disclosed herein can come in many shapes, sizes, and geometries. In some embodiments, the drug delivery device can be circular, square, rectangular, oval, triangular, diamond shaped, polygon shaped (e.g., pentagon, hexagon, octagon, etc.), arced, trapezoidal, star shaped, or a variety of other shapes and sizes. In some embodiments, the drug delivery device can be tubular, cylindrical, cone shaped, pyramidal, triangular prism shaped, cube shaped, spherical, screw-shaped, rectangular prism-shaped, or a variety of other shapes and sizes. For example, FIGS. 1A-D illustrate a rectangular prism-shaped drug delivery device 100 and FIGS. 2A-C illustrate cylindrical drug delivery device 200.

In some embodiments, the drug delivery device can include a plurality of layers. In some embodiments, the drug delivery device can include at least one biodegradable layer. At least one layer or multiple layers can include an API which can be released in vivo by polymer biodegradation. In some embodiments, at least one second layer or multiple layers can be free of any API.

In some embodiments, the drug delivery device can include a body. In some embodiments, the body of the drug delivery device can be flexible or rigid. In some embodiments, the body can include a plurality of layers. In some embodiments, drug delivery device can include at least one API layer or multiple API layers. The API layer can include API embedded within and/or on the surface of the at least one API layer. In some embodiments, the plurality of layers (i.e., all other layers) of the drug delivery device can also be API layers. These layers can include the same or different API. In some embodiments, at least one of the layers or multiple layers of the drug delivery device can be a non-API layer.

For example, the drug delivery device can include body 204. In some embodiments, the body can include at least one API layer 101 (e.g., an outer layer). The API layer can include API embedded within and/or on the surface of the at least one API layer 101. In some embodiments, the plurality of layers (e.g., all other layers) of the drug delivery device can also be API layers. For example, layers 102 (e.g., an inner layer), 103, 104, and 105 can be layers that include API embedded within and/or on the surface of the layer. These layers can include the same or different API. In some embodiments, at least one of the layers of the drug delivery device can be a non-API layer. For example, middle layer 103 can be a non-API layer to provide some additional structural support and/or strength to the overall drug delivery device. In use, the drug delivery device can be implanted within a solid tumor (i.e., intratumorally) and/or adjacent to a solid tumor and the API layer(s) can release the API within or adjacent to the tumor during degradation as shown by the arrows 301 in FIGS. 1A-C and 2A-C. As such, the API layer(s) can release the API during degradation multi-directionally and/or omnidirectionally (i.e., in every direction) within the target tissue. In some embodiments, the targeted tissue can be a cancerous tumor (e.g., a solid tumor). For example, the targeted tissue can be a tumor on or in the pancreas, biliary system, gallbladder, esophageal system, liver, stomach, peritoneum, small bowel, lung, colon, or metastasis from a primary tumor.

In some embodiments, the outer layers of the device (e.g., layers 101 and 105) can be non-API layers. In some embodiments, the body can be coated with a non-API coating or can be covered on all sides with non-API layers (e.g., a non-API shell). For example, in such an embodiment, the cross section of FIG. 1C would include a non-API layer(s) or coating around the perimeter of the cross section (not shown). In such an embodiment, an API layer(s) can be on the inside of the body of the drug delivery device such that drug will not be accidentally released, lost, and/or damaged (e.g., due to inadvertent scraping and/or damage of device during placement) until the non-API coating shell has dissolved first. In other words, the drug delivery device can have a protective non-API layer(s)/coating so that API layers are protected from any outside elements. For example, in some embodiments, all sides (e.g., top, bottom, front, back, each ends) of the drug delivery device can have a non-API coating/layer/shell. In some embodiments, the non-API layer/coating/shell can include biodegradable polymers (disclosed herein) that have a slower, faster, or the same degradation rate than the biodegradable polymers of the API layer(s). In some embodiments, the outer layers may degrade faster than the inner layers of the drug delivery device regardless of the degradation rates of the layers as the outer layers are in contact with the environment that degrades the biodegradable polymer, whereas the inner layers can be protected from such an environment. In some embodiments, the outer layers/shell of the device can be API layers so that the API(s) can readily be released once implanted in the target tissue without waiting for a non-API layer to degrade.

In some embodiments, the body of the drug delivery device can include at least one API layer and/or at least one non-API layer. As described above, the at least one API layer can be an outer layer, inner layer, shell layer, core, and/or core layer. In addition, the at least one non-API layer can be an outer layer, inner layer, shell layer, core, and/or core layer. In some embodiments, a core, core layer, outer layer, inner layer, and/or shell layer can have the compositions of any API layer or non-API layer disclosed herein. For example, FIGS. 2A-C illustrate non-API core 202 with API outer layer 201. In some embodiments, the core or core layer can provide strength to the drug delivery device as it is inserted in the patient. As shown in FIG. 2B, core 202 can provide longitudinal strength to the drug delivery device for implanting into and/or adjacent to a target tissue site of the patient. Although FIG. 2C only illustrates two layers, the drug delivery device disclosed herein can have additional layers such as API layers, non-API layers, or a combination thereof. In some embodiments, drug delivery device 200 can include API layer 201 (i.e., an outer layer) and non-API layer 202 (i.e., a core or core layer). The API-containing layer can include API embedded within and/or on the surface of API layer 201. In some embodiments, an API layer can surround at least a portion of the non-API core or core layer. In some embodiments, a non-API layer can surround at least a portion of an API core or core layer. In some embodiments, the drug delivery device may have a hollow core as discussed below. The hollow core may be used to help guide and deliver the drug delivery device to its target tissue.

In some embodiments, the drug delivery devices can be made by a solvent casting method. As described in more detail below, at least one biodegradable polymer and at least one API can be dissolved in a solvent and cast in a mold and/or on a substrate. In some embodiments, after drying, the layer can be removed from the mold and/or substrate and this process can be repeated to create as many API layers as required for the drug delivery device. In some embodiments, the layer can remain on the substrate for further processing such as lamination and/or cutting as described herein. In some embodiments, a second layer (i.e., a non-API layer) having at least one biodegradable polymer can be dissolved in a solvent, cast in a mold and/or on a substrate, dried, and/or removed from the mold and/or substrate. This process can be repeated to create as many API layers as required for the drug delivery device. In some embodiments, any combination and order of API and/or non-API layers can be laminated together to form the drug delivery device or laminated together then cut (e.g., die cut) to form the drug delivery device.

In some embodiments, a second layer having at least one biodegradable polymer (and optionally API) can be cast on top of the first layer and then dried (and repeated) to form the device. In some embodiments, the non-API layer (e.g., a core or core layer) can be dissolved in the solvent and cast in the mold and/or on the substrate prior to an API layer (e.g., outer layer or shell layer) being cast on top of the non-API layer. This process can be repeated for as many additional API and/or non-API layers as necessary.

In some embodiments, the drug delivery device can be made via a continuous process. In some embodiments, the drug delivery devices disclosed herein can be made by continuous processes such as those employed by the polymer film production industry (e.g., extrusion, continuous film evaporation, etc.).

In some embodiments, such as those shown in FIGS. 1A-C and 2A-C, the drug delivery device is configured to be inserted into and/or adjacent to target tissue site of a patient using standard open, laparoscopic, endoscopic (e.g., bronchoscopic), percutaneous, or robotic surgical equipment. In some embodiments, the drug delivery device can be inserted through a working channel of an endoscope (e.g., a bronchoscope), trocar, or needle (for percutaneous delivery). For example, the drug delivery device can be guided to the target tissue site through a working channel of an endoscope (or robotic endoscope). In some embodiments, the device is flexible such that it can be delivered to the target tissue via any of the above administration routes.

Because the drug delivery device can be flexible to be configured to be inserted through a working channel of an endoscope, trocar, etc., the diameter, width, and/or height of the drug delivery device (of body and/or tip) can be between about 0.1-20 mm, about 0.25-20 mm, 0.25-12 mm, 0.5-12 mm, about 0.5-10 mm, about 0.5-8 mm, about 0.5-5 mm, about 0.5-3 mm, about 1-3 mm, or about 2 mm. In some embodiments, the length of the body of the drug delivery device can be about 0.1-20 mm, about 0.5-15 mm, about 1-15 mm, about 3-12 mm, about 4-11 mm, or about 5-10 mm. In some embodiments, the device can have a smaller diameter, width, and/or height to be inserted percutaneously.

In some embodiments, the drug delivery device can include a tip 205. In some embodiments, the drug delivery device may not include a tip. In some embodiments, the tip of the drug delivery device can be flexible or rigid. In some embodiments, the tip can be connected to the distal end of the body of the drug delivery device. In some embodiments, the tip and the body can be an integral unit. As the drug delivery device is being guided to the tissue site of the patient, the drug delivery device can be guided tip first. In some embodiments, the tip can have at least one vertex at its distal end. The at least one vertex can allow the tip (and the drug delivery device) to penetrate into and/or adjacent to the target tissue site (e.g., solid tumor). In some embodiments, the at least one vertex can be sharp. This can allow the drug delivery device to be implanted into and/or adjacent to the target tissue site of the patient tip first. In some embodiments, the drug delivery device does not include a tip.

In some embodiments, the tip can be configured to retain or hold the drug delivery device in the target tissue site. For example, in some embodiments, the tip can include tissue retainer(s) or tissue retaining mechanism(s) 206. The tissue retaining mechanism(s) can be configured to retain or hold the drug delivery device in the target tissue site (i.e., in the solid tumor). In some embodiments, the tissue retaining mechanism(s) can extend outwardly from the tip. In some embodiments, the tissue retaining mechanism(s) can extend radially outward from the drug delivery device. In some embodiments, the tissue retaining mechanism(s) can extend outward from the drug delivery device (or tip) and back towards the body or proximal end of the drug delivery device. In some embodiments, the tissue retaining mechanism(s) can include edges, barbs, a lip(s) or similar other aspects. In some embodiments, the edges, barbs, lip(s), or similar other aspects can be sharp. In some embodiments, the tip with tissue retaining mechanism(s) can be shaped similar to that of a broadhead arrow. As such, the tip (with tissue retaining mechanism(s)) can be configured to penetrate the target tissue site, embed the drug delivery device within or adjacent to the target tissue site, and retain/hold the drug delivery device within or adjacent to the target tissue site as the API is released from the API layer(s). In some embodiments, the body of the drug delivery device can include a tissue retaining mechanism(s) such as a non-API or API layer(s) of the body.

In some embodiments, the tip can also include a biodegradable polymer. In some embodiments, the biodegradable polymer of the tip has a slower degradation rate than the biodegradable polymer in the API layer(s) such that the tip can retain the drug delivery device in place within the tumor as the API is released from the API layer(s) by in vivo degradation. Similar to the API and/or non-API layers, the tip can be configured/tuned to have a specific desired degradation rate. In some embodiments, the tip can be made out of any of the compositions disclosed herein for an API layer and/or a non-API layer. In some embodiments, the tip can have the same composition of any non-API layer disclosed herein. In some embodiments, the tip and body of the device can be integral components or created separately and later attached/adhered/connected to one another. In some embodiments, the tip can be created the same way any API and/or non-API disclosed herein can be created (e.g., solvent casting, lamination, cutting, etc.). In addition, because the tip can be made of a biodegradable material, the entirety of the drug delivery device can be degradable such that no subsequent surgery may be required to remove an aspect of the device. In some embodiments, the thickness of the layers of the drug delivery device and/or size of the tip can be selected based on the desired degradation/API release kinetics.

In some embodiments, when implanting the drug delivery device into and/or adjacent to the target tissue site, the drug delivery device can be configured to disengage or release from a component (e.g., a tool that can deliver the device such as shaft 203 that can be flexible). In some embodiments, for delivery, a flexible shaft (e.g., a distal end of the flexible shaft) can be connected to the body (e.g., a proximal end of the body) of the drug delivery device. The drug delivery device with flexible shaft can then be inserted into a working channel of an endoscope (e.g., standard or robotic endoscope) to be implanted into and/or adjacent to the target tissue site of a patient. In some embodiments, the proximal end of the flexible shaft can be connected or attached to a component of an endoscope, another tool used to guide the drug delivery device to the target tissue site, and/or a robotic component. In some embodiments, disengaging or releasing the drug delivery device from the delivery tool (e.g., flexible shaft) can include twisting the delivery tool with respect to the drug delivery device. This motion can release/disengage the drug delivery device from the delivery tool (e.g., flexible shaft). In some embodiments, this motion can fatigue the portion of the drug delivery device connected to the delivery tool and separate the tool from the drug delivery device.

In some embodiments, the drug delivery device may have a hollow core such that the drug delivery device can act as a shaft or shank for a central pin, peg, and/or mandrel (e.g., a flexible shaft 203) to deliver the device to the target tissue site such as drug delivery device shown in FIGS. 2E-F. In some embodiments, the pin, peg, and/or mandrel can then be removed from the drug delivery device such that the drug delivery device is left in its target tissue site. This would be similar to that of a rivet fastening mechanism or a stent deployment. In some embodiments, a tip of the drug delivery device could be attached to a deployment mandrel through the core of the drug delivery device. When the mandrel is removed, the mandrel can break off or be disconnected from the tip similar to that of a pop rivet mechanism. In some embodiments, the hollow core of the drug delivery device may have an inner diameter configured to pass or translate a delivery tool (e.g., flexible shaft) through the hollow core. For example, the hollow core may have a diameter of at least about 0.1 mm, at least about 0.5 mm, at least about 1 mm, or at least about 2 mm. In some embodiments, the hollow core can have a diameter of at most 10 mm, at most 5 mm, at most 2 mm, or at most 1 mm. For example, in some embodiments, a distal end of a flexible shaft 203 can be attached/connected/adhered/etc. to the distal end of the drug delivery device (e.g., the tip 205) through the hollow core of the drug delivery device. This can give longitudinal support, strength, and/or rigidity to the drug delivery device for implantation into and/or adjacent to a target tissue site. In some embodiments, the proximal end of the flexible shaft can be connected or attached to a component of an endoscope, another tool used to guide the drug delivery device to the target tissue site, and/or a robotic component. In some embodiments, disengaging or releasing the drug delivery device from the delivery tool (e.g., flexible shaft) can include twisting or popping the delivery tool with respect to the drug delivery device. This motion can release/disengage the drug delivery device from the delivery tool (e.g., flexible shaft). In some embodiments, this motion can fatigue the portion of the drug delivery device connected to the delivery tool and separate the tool from the drug delivery device.

In some embodiments, the drug delivery device can be attached to forceps 210 (e.g., standard lung biopsy forceps) as shown in FIG. 2D that can implant the drug delivery device into and/or adjacent to the target tissue site. In some embodiments, the forceps can be biopsy forceps (e.g., clamshell forceps). For example, a mouth of the forceps can be attached to where disengagement mechanism 207 is located and the forceps can be used to place the drug delivery device at its target location. Once the drug delivery device is in place, it can be released by opening the forceps.

In some embodiments, the drug delivery device can be housed in an applicator. The applicator can include a plunger that can push the drug delivery device out of the applicator housing and into and/or adjacent to the target tissue site (i.e., similar to that of a tampon). The applicator can then be removed after the drug delivery device is employed.

In some embodiments, the body can include a disengagement mechanism 207 towards a proximal end of the body. In some embodiments, the body can include a disengagement mechanism towards a distal end of the body. The disengagement mechanism can be configured to disengage the drug delivery device from the delivery tool. After the drug delivery device is implanted/embedded in the target tissue (e.g., solid tumor), the delivery tool (e.g., flexible shaft, forceps, etc.) can be withdrawn (in some embodiments from the working channel of the endoscope) leaving the drug delivery device in the target tissue. In some embodiments, the tip of the drug delivery device can include the disengagement mechanism(s) such as that shown in FIG. 2E.

In some embodiments, drug delivery device can include anatomical marker(s) 208. In some embodiments, the anatomical marker(s) can be in an API layer and/or non-API layer of the device. In some embodiments, the anatomical marker can be in a body and/or tip of the drug delivery device. In some embodiments, the anatomical marker can be used by a physician to identify the drug delivery device in the patient using a wide variety of imaging techniques. The physician can then monitor the progress and/or location of the drug delivery device with respect to the tumor. In some embodiments, the anatomical marker can be a radiopaque marker, x-ray markers, lead markers, or similar marker(s). In some embodiments, an anatomical market can be embedded into a component (e.g., a layer, the body, and/or the tip) after that component has already been created.

As stated above, the drug delivery device can be guided to the targeted tissue site via open surgery, laparoscopically, robotically, percutaneously, endoscopically, or combinations thereof. Once at the target tissue site, a camera or monitoring of anatomical markers by other means can be utilized to make sure that the drug delivery device is properly oriented. In some embodiments, a surgical camera can be utilized to make sure that the (at least one vertex of the) tip of the drug delivery device is pointed toward the targeted tissue site.

Once the correct placement and orientation of the drug delivery device is confirmed, the drug delivery device can be inserted or implanted into and/or adjacent to the target tissue site. For example, the drug delivery device can be implanted such that at least part of the drug delivery device is within or adjacent to the target tissue site (e.g., solid tumor). In some embodiments, the entirety of the drug delivery device can be implanted within the target tissue site. In some embodiments, after the drug delivery device is implanted in or adjacent to the target tissue, the delivery tool (e.g., flexible shaft, forceps, etc.) can be disengaged from the drug delivery device and then removed from the patient leaving the drug delivery device.

API Layer(s)

In some embodiments, a drug delivery device can include at least one or multiple API containing layers. In some embodiments, the at least one API containing layer can be an outer layer, inner layer, core, core layer and/or shell layer of the drug delivery device. In some embodiments, an API layer can include at least one active pharmaceutical ingredient (API) and at least one biodegradable polymer. In some embodiments, the API layer can include more than one biodegradable polymer. In some embodiments, an API layer can include more than one API. As explained above, an API layer can be configured to provide controlled release of the API by in vivo degradation of the biodegradable polymer at and/or in the target tissue site. In some embodiments, an API layer can also include one or more pharmaceutically acceptable excipients.

In some embodiments, the biodegradable polymer can be any suitable biodegradable polymer known in the art. For example, the biodegradable polymers can include synthetic polymers selected from poly(amides), poly(esters), poly(anhydrides), poly(orthoesters), polyphosphazenes, pseudo poly(amino acids), poly(glycerol-sebacate), copolymers thereof, and mixtures thereof. In addition, the biodegradable polymers may be formed from poly(lactic acids), poly(glycolic acids), poly(lactic-co-glycolic acids), poly(caprolactones), and mixtures thereof. In some embodiments, the biodegradable polymer can be poly(lactic-co-glycolic acid) (PLGA). In some embodiments, the PLGA can be PLGA with various lactic acid to glycolic acid ratios such as PLGA 50:50, PLGA 60:40, PLGA 65:35, PLGA 70:30, PLGA 75:25, PLGA 80:20, PLGA 85:15, PLGA 90:10, or other various ratios of PLGA. In some embodiments, the biodegradable polymer in an API containing layer includes PLGA 50:50.

PLGA can undergo degradation by hydrolysis or biodegradation through cleavage of its backbone ester linkages into oligomers followed by monomers. The lactide-glycolide ratio dictates the degradation rate of the PLGA in aqueous media (e.g., water and water containing environments such as inside a human or animal's anatomy). In general, the higher lactic acid content or lactide content, the lower the degradation behavior of the PLGA as lactide has hydrophobic properties which can prevent water from hydrolyzing the ester bonds in PLGA. As such, PLGA 50:50 degrades faster in water-containing environments than PLGA 65:35, which degrades faster than PLGA 75:25.

In some embodiments, an API containing layer includes at least about 50 wt. %, at least about 60 wt. %, at least about 70 wt. %, at least about 73 wt. %, at least about 75 wt. %, at least about 80 wt. %, at least about 85 wt. %, or at least about 90 wt. % biodegradable polymer. In some embodiments, an API containing layer includes at most about 95 wt. %, at most about 90 wt %, at most about 85 wt. %, at most about 80 wt. %, at most about 77 wt. %, or at most about 75 wt. % biodegradable polymer. In some embodiments, an API containing layer includes about wt. %, about 70-95 wt. %, about 75-90 wt. %, about 75-89 wt. %, or about 79-89 wt. % biodegradable polymer.

In some embodiments, the API may be an active pharmaceutical ingredient for the treatment of human or veterinary diseases. APIs may be one or more of antibacterial agents, antifungal agents, antiprotozoal agents, antiviral agents, labor-inducing agents, spermicidal agents, prostaglandins, steroids and microbicides, proteins/peptides and vaccine antigens.

Suitable APIs include, without limitation: analgesics and anti-inflammatory agents (e.g., ibuprofen), antacids, anthelmintics, anti-arrhythmic agents, anti-bacterial agents, anti-coagulants, anti-anxiety anti-depressants, anti-diabetics, anti-diarrhoeals, anti-epileptics, anti-fungal agents, anti-gout agents, anti-hypertensive agents, anti-malarials, anti-migraine agents, anti-muscarinic agents, anti-neoplastic agents and immunosuppressants, anti-protazoal agents, anti-rheumatics, anti-thyroid agents, antivirals, anxiolytics, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, lipid regulating agents, local anesthetics, neuro muscular agents, nitrates and anti-anginal agents, nutritional agents, opioid analgesics, oral vaccines, proteins, peptides and recombinant drugs, sex hormones and contraceptives, spermicides, stimulants, smoking cessation products and combinations thereof.

In some embodiments, the API can be a chemotherapeutic agent such as any drug formulation effective to treat cancer by inhibiting the growth, invasiveness of malignant cells, and/or inducing cytotoxicity by apoptosis or necrosis of malignant cells. The chemotherapeutic agent can be a taxane or platinum drug. In some embodiments, the chemotherapeutic agent includes an MEK inhibitor, a KRAS inhibitor, a PI3K inhibitor, a Hedgehog inhibitor, a Wnt inhibitor, or a combination thereof. In some embodiments, the chemotherapeutic agent can interfere with the mTOR or NfKb pathways. In some embodiments, the chemotherapeutic agent includes STING agonist compounds. In some embodiments, the chemotherapeutic agent can interfere with the STING pathway. In some embodiments, the chemotherapeutic agent includes genetic material such as mRNA, siRNA, etc. In some embodiments, the chemotherapeutic agent can be siRNA-Alnylam type therapies.

In some embodiments, the API can include a vaccinal antigen. In some embodiments, the API can be an mRNA vaccinal antigen such as an mRNA cancer vaccinal antigen.

The API may be a single active pharmaceutical ingredient, such as a single chemical entity, or it may be a mixture of several active pharmaceutical ingredients. The active pharmaceutical ingredient may be of any of the many categories of active pharmaceutical ingredients. The active pharmaceutical ingredient may be selected from, but is not limited to, the group consisting of paclitaxel, gemcitabine, nab-paclitaxel, 5-fluorouracil, oxaliplatin, irinotecan, docetaxel, vinorelbine, etoposide, mitomycin-C, cisplatin/carboplatin, fluorouracil, methotrexate, TAS-102, or combinations thereof. In some embodiments, the API is paclitaxel. In some embodiments, the paclitaxel is from Phyton Biotech.

In some embodiments, the API can be an API for targeted therapy such as bevacizumab, ramucirumab, erlotinib, afatinib, gefitinib, osimertinib, dacomitinib, crizotinib, ceritinib, alectinib, brigatinib, lorlatinib, dabrafenib, trametinib, sunitinib, regorafenib, or combinations thereof. In some embodiments, the API can be an API for immunotherapies such as nivolumab, pembrolizumab, atezolizumab, durvalumab, ipilimumab, or combinations thereof.

In some embodiments, the API can be acyclovir, fluconazole, progesterone and derivatives thereof, nonoxylenol-9, terbutaline, lidocaine, testosterone and derivatives, dinoprostone, lactobacillus, estrogen and derivatives, naphthalene2-sulfonate, lesmitidan, doxycycline, droxidopa, sapropterin, butoconazole, clindamycin nitrate/phosphate, neomycine sulfate, polymyxin sulfate, nystatin, clotrimazole, dextrin sulphate, glyminox, miconazole nitrate, benzalkonium chloride, sodium lauryl sulphate, tenofovir, insulin, calcitonin, danazol, ibuprofen, acetaminophen, cefpodoxime proxetil, desloratadine, dextromethorphan, diphenhydramine hydrochloride, vitamins and/or minerals, adipic acid, ascorbic acid, macrolide antibiotics, NS-AIDS, cefuroxime axetil, amobarbital, ciprofloxacin hydrochloride, sildenafil citrate, pinaverium bromide, propantheline bromide, triprolidine Hcl, dimenhydrinate, cefeanel daloxate HCl, Enoxacin, Sparfloxacin, aspirin, famotidine, amoxycilin trihydrate, morphine HCl, amiprilose HCl, terfenadine, beclamide, clarithromycin, roxithromycin, nizatidine, cetraxate HCl, ciprofloxacin, bifemelene HCl, Cefuroxime axetil, pirienzepine and/or oxyburynin, diclofenac, nicorandil, levofloxacin, acriflavine, leuprorelin acetate, metronidazole, benzydamine hydrochloride, chloramphenicol, oxybutynin, ethinyl estradiol, prostaglandins, insulin, calcitonin and combinations thereof. The active pharmaceutical ingredient may also be vaccine antigen such as those for the treatment of Hepatitis B, HIV, HPV, Chlamydia, gonococcal infections.

APIs may include salts, esters, hydrates, solvates and derivatives of any of the foregoing active ingredients. Suitable derivatives are those that are known to skilled persons to possess the same activity as the active ingredient though the activity level may be lower or higher. In some embodiments, the API can be in the form of microspheres. In some embodiments, the microspheres can release the API. In some embodiments, the API is encapsulated within microspheres.

When present, an API is employed in the formulation in a therapeutically effective amount that is necessary to provide the dosage required, typically for producing at least one physiological effect as established by clinical studies. One of ordinary skill in the art can readily determine an appropriate amount of an active pharmaceutical ingredient to include in the drug delivery device made according to the present disclosure.

In some embodiments, an API containing layer includes at least about 1 wt. %, at least about 2 wt. %, at least about 3 wt. %, at least about 5 wt. %, at least about 7 wt. %, at least about 8 wt. %, at least about 9 wt. %, at least about 10 wt. %, at least about 11 wt. %, at least about 12 wt. %, at least about 13 wt. %, at least about 14 wt. %, or at least about 15 wt. % API. In some embodiments, an API containing layer includes at most about 30 wt. %, at most about 25 wt. %, at most about 20 wt. %, at most about 18 wt. %, at most about 15 wt. %, at most about 14 wt. %, at most about 13 wt. %, at most about 12 wt. %, at most about 11 wt. %, at most about 10 wt. %, at most about 9 wt. %, at most about 8 wt. %, at most about 7 wt. %, or at most about 5 wt. % API. In some embodiments, an API containing layer includes about 1-30 wt. %, about 1-25 wt. %, about 5-25 wt. %, about 10-20 wt. %, about 12-18 wt. %, about 12-15 wt. %, about 13-14 wt. %, about 1-15 wt. %, about 5-15 wt. %, about 7-12 wt. %, about 8-12 wt. %, or about 7.5-11 wt. % API. In some embodiments, an API containing layer can include about 0.01-500 mg, about 0.01-50 mg about 0.1-50 mg, or about 0.25-50 mg.

To prepare an API layer in some embodiments of the drug delivery devices disclosed herein, a solution of a biodegradable polymer and an API can be formed. Specifically, an amount of biodegradable polymer can be dissolved in a solvent. The biodegradable polymer/solvent solution can be stirred and/or heated to help dissolve the polymer in the solvent. In some embodiments, the solvent can be acetone, chloroform, tetrahydrofuran, ethyl acetate, methyl acetate, xylene, toluene, methyl ethyl ketone, methylene chloride, isopropyl alcohol, methyl isobutyl ketone, methyl propyl ketone, trichloroethylene, other substitutes for acetone, or combinations thereof. In some embodiments, the biodegradable polymer and solvent solution can be about 0.1-0.3 g biodegradable polymer, about 0.15-0.25 g biodegradable polymer, about 0.175-0.225 g biodegradable polymer, about 0.19-0.21 g biodegradable polymer, about 0.198-0.202 g biodegradable polymer, or about 0.2 g biodegradable polymer per 1 mL solvent. In other words, 20 g biodegradable polymer in 100 mL of solvent would be a biodegradable polymer and solvent solution of 0.2 g biodegradable polymer per 1 mL solvent. The above concentrations can also be true for the biodegradable polymer/API solution discussed below. For example, the biodegradable polymer/API solution can have about g biodegradable polymer, about 0.15-0.25 g biodegradable polymer, about 0.175-biodegradable polymer, about 0.19-0.21 g biodegradable polymer, about 0.198-biodegradable polymer, or about 0.2 g biodegradable polymer per 1 mL solvent.

In some embodiments, after the biodegradable polymer is dissolved in the solvent, the API can be added to the solution. In some embodiments, the API and biodegradable polymer can be added simultaneously to the solvent. In some embodiments, the API can be added to the solvent prior to the addition of the biodegradable polymer. In some embodiments, the API can be added to a solvent to form a first solution, the biodegradable polymer can be added to a solvent to form a second solution, and the first and second solutions can be combined to form the biodegradable polymer/API solution. In some embodiments, the API can be in the form of microspheres when incorporating the API to a biodegradable polymer layer. In some embodiments, the microspheres can help prevent the API from dissolving and/or deteriorating in the polymer/solvent solution.

In some embodiments, the biodegradable polymer/API solution can have about API, about 0.01-0.045 g API, about 0.015-0.045 g API, about 0.02-0.04 g API, about 0.025-0.04 g API, or about 0.03-0.04 g API per 1 mL solvent. The above concentrations are also true for an API and solvent solution (without the biodegradable polymer). The biodegradable polymer/API solution can be stirred and/or heated until the API is well mixed and/or dissolved in the solvent.

In some embodiments, after the biodegradable polymer/API solution is formed, the solution can be added to a mold and/or a substrate. In some embodiments, the substrate can be a release film or liner such as a medical release liner. In some embodiments, the substrate can be made form a polymer (e.g., polyester) or paper substrate and could be coated with silicone or PTFE. In some embodiments, the mold can be any container used to give shape to the API layer when it is formed. As such, the mold can be circular, square, rectangular, oval, triangular, diamond shaped, polygon shaped (e.g., pentagon, hexagon, octagon, etc.), arced, trapezoidal, star shaped, tubular, cylindrical, cone shaped, pyramidal, triangular prism shaped, cube shaped, spherical, rectangular prism shaped, or a variety of other shapes and sizes. Besides shape, the mold can also dictate the size (i.e., width, length, diameter, height/thickness, etc.) of the layer.

In some embodiments, the mold can dictate the shape of the final API layer. In other embodiments, the mold forms a precursor API layer which is later modified (e.g., lamination and cutting) to form the final API layer of the drug delivery device. In some embodiments, the biodegradable polymer/API solution can be added to a circular mold. In some embodiments, the mold can be an evaporation dish, a petri dish, or the like. In addition, the amount added to the mold can depend on the desired thickness and/or API concentration of the API layer. In addition, the amount added to the mold can depend on the desired API release rate of the API layer. In some embodiments, about 1-20 mL, about 1-10 mL, about 4-6 mL, or about 5 mL of the biodegradable polymer/API solution can be added to the mold.

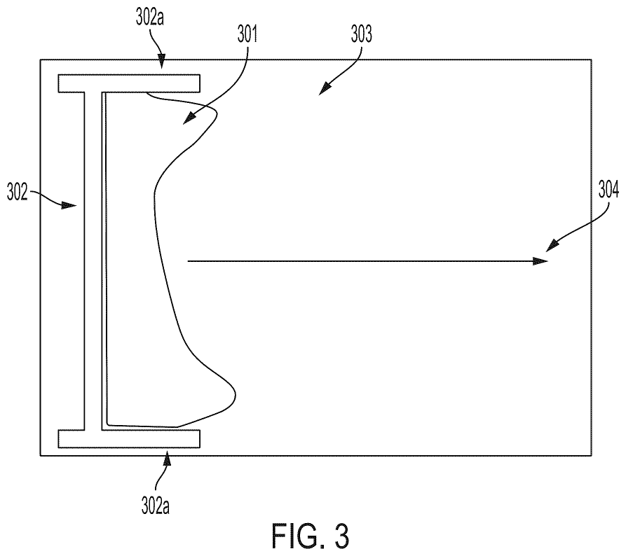

In some embodiments, the biodegradable polymer/API solution is added to the substrate. As stated above, in some embodiments, the substrate can be a release liner and the biodegradable polymer/API solution can be added to the release side of a release liner. In some embodiments, a film applicator and/or a spiral bar coater can be used to create reproducible wet layers of the biodegradable polymer/API solution of a defined thickness. In some embodiments, the film applicator and/or spiral bar coater can be placed on the flat substrate with the desired thickness chosen. In some embodiments, the biodegradable polymer/API solution can then be added to the substrate. In some embodiments, the biodegradable polymer/API solution can be added in front of the film applicator and/or spiral bar coater and/or in a reservoir of the film applicator and/or spiral bar coater. In some embodiments, consistent pouring speed of the biodegradable polymer/API solution can create a straighter leading edge. In some embodiments, starting with more volume of the biodegradable polymer/API solution at the edges near the feet of the film applicator and/or spiral bar coater can create a straighter trailing edge. In some embodiments, the biodegradable polymer/API solution 301 can be added between the feet 302a of the film applicator 302 and/or spiral bar coater as shown in FIG. 3. In some embodiments, once the biodegradable polymer/API solution is in place, the film applicator and/or spiral bar coater can be moved across the substrate 303 (i.e., the drawdown) at a steady speed in a given direction 304.

In some embodiments, moving the film applicator and/or spiral bar coater across the substrate can guide the biodegradable polymer/API solution through a gap of the film applicator and/or spiral bar coater and spread the biodegradable polymer/API solution across the substrate, thereby creating a wet layer of the biodegradable polymer/API solution on the substrate of the desired thickness. In some embodiments, the film applicator and/or spiral bar coater can be a manual or automatic film applicator. In some embodiments, the film applicator can be a baker-type applicator, a bird-type applicator, a reservoir applicator, and/or a micrometric applicator, among others.

In some embodiments, the thickness of the wet layer of biodegradable polymer/API solution on the substrate can be at least about 0.1 mm, at least about 0.25 mm, at least about 0.5 mm, at least about 0.75 mm, at least about 1 mm, at least about 1.25 mm, at least about 1.5 mm, at least about 1.75 mm, or at least about 2 mm thick. In some embodiments, the thickness of the wet layer of biodegradable polymer/API solution on the substrate can be at most about 5 mm, at most about 4 mm, at most about 3 mm, at most about 2 mm, at most about 1.5 mm thick, or at most about 1 mm thick.

In some embodiments, once spread across the substrate, the biodegradable polymer/API solution can be dried. This drying can cause solvent to evaporate, thereby leaving a solidified layer that includes biodegradable polymer, API, and some remaining solvent. In some embodiments, remaining solvent in the layer can be important because it allows the layer(s) to retain flexibility and not crack during subsequent processing. As such, an API layer can also include a small amount of solvent, which will be discussed in further detail below. In some embodiments, once in the mold, biodegradable polymer/API solution can be dried.

In some embodiments, the biodegradable polymer/API solution can be dried in air, nitrogen, or other gases at at least room temperature (e.g., 20-25° C.). In some embodiments, the biodegradable polymer/API solution can be dried at at least about 20° C., at least about 25° C., or at least about 30° C. In some embodiments, the biodegradable polymer/API solution can be dried in an environment with a humidity of less than about 30%, less than about 25%, less than about 20%, less than about 15%, less than about 10%, less than about 5%, or less than 1%. In some embodiments, the drying can be for at least about 1 minute, at least about 5 mins, at least about 10 mins, at least about 15 mins, at least about 30 mins, at least about 1 hr, at least about 12 hours, or at least about 1 day. In some embodiments, the drying can be at most 6 days, at most 5 days, at most about 2 days, at most about 1 day, at most about 12 hours, at most about 6 hours, at most about 1 hour, or at most about 30 mins.

### Non-API Layer(s)

In some embodiments, a drug delivery device can include at least one or multiple non-API containing layers. In some embodiments, the at least one non-API containing layer can be an outer layer, inner layer, core, core layer, and/or shell layer of the drug delivery device. In some embodiments, a non-API layer can be free from an API. In some embodiments, the non-API layer can include at least one or multiple biodegradable polymer. As explained above, in some embodiments, a non-API layer can act as a backing layer or supporting layer to an API layer and/or the drug delivery device to provide strength (e.g., longitudinal strength) to the drug delivery device.

In some embodiments, a non-API layer can include a biodegradable polymer with a slower degradation rate than the biodegradable polymer in an API layer. As explained below, PLGA 50:50 can be the biodegradable polymer in an API layer and PLGA 75:25 can be a biodegradable polymer in a non-API layer as PLGA 75:25 degrades slower than the PLGA 50:50. In some embodiments, the non-API layer can include a biodegradable polymer with the same or faster degradation rate than the biodegradable polymer in an API layer. In some embodiments, a non-API layer can also include one or more pharmaceutically acceptable excipients.

As an example, the biodegradable polymer in an API layer may degrade over the course of about 4 weeks releasing the API into and/or adjacent to the targeted tissue. The biodegradable polymer in a non-API layer may degrade in a longer period of time (e.g., 10 weeks). Thus, in some embodiments, the non-API layer can be used to provide strength, structure, and/or support for an API layer and/or the drug delivery device in its targeted location while an API layer(s) degrades to release the API.

After an API layer(s) completely degrades and the API has been completely released to the targeted tissue, a non-API layer(s) can completely degrade to be absorbed by the body of the patient. In some embodiments, a portion of a non-API layer can degrade while an API layer is degrading, but the degradation rate of the non-API layer may be slower than an API layer's degradation rate.

In some embodiments, the biodegradable polymer can be any suitable biodegradable polymer known in the art. As explained above, a biodegradable polymer in a non-API layer may a slower, faster, or the same degradation rate as a biodegradable polymer in an API layer. For example, the biodegradable polymers can include synthetic polymers selected from poly(amides), poly(esters), poly(anhydrides), poly(orthoesters), polyphosphazenes, pseudo poly(amino acids), poly(glycerol-sebacate), copolymers thereof, and mixtures thereof. In addition, the biodegradable polymers may be formed from poly(lactic acids), poly(glycolic acids), poly(lactic-co-glycolic acids), poly(caprolactones), and mixtures thereof. In some embodiments, the biodegradable polymer can be poly(lactic-co-glycolic acid) (PLGA). In some embodiments, the PLGA can be PLGA with various lactic acid to glycolic acid ratios such as PLGA 50:50, PLGA 60:40, PLGA 65:35, PLGA 70:30, PLGA 75:25, PLGA 80:20, PLGA 85:15, PLGA 90:10, or other various ratios of PLGA. In some embodiments, the biodegradable polymer in a non-API containing layer includes PLGA 75:25.

In some embodiments, a non-API containing layer includes at least about 70 wt. %, at least about 75 wt. %, at least about 80 wt. %, at least about 85 wt. %, at least about 90 wt. %, or at least about 95 wt. % biodegradable polymer. In some embodiments, a non-API containing layer includes at most about 99.9 wt. %, at most about 99 wt. %, at most about 98 wt %, at most about 97 wt. %, at most about 95 wt. %, or at most about 90 wt. % biodegradable polymer. In some embodiments, a non-API containing layer includes about 80-99.9 wt. %, about 85-98 wt. %, about 86-97 wt. %, or about 88-96 wt. % biodegradable polymer.

To prepare a non-API layer for the drug delivery device, a second solution of a biodegradable polymer can be formed. Specifically, an amount of biodegradable polymer can be dissolved in a solvent. The second biodegradable polymer/solvent solution can be stirred and/or heated to help dissolve the polymer in the solvent. In some embodiments, the solvent can be acetone, chloroform, tetrahydrofuran, ethyl acetate, methyl acetate, xylene, toluene, methyl ethyl ketone, methylene chloride, isopropyl alcohol, methyl isobutyl ketone, methyl propyl ketone, trichloroethylene, other substitutes for acetone, or combinations thereof. In some embodiments, the biodegradable polymer and solvent solution can be about 0.1-0.3 g biodegradable polymer, about 0.15-0.25 g biodegradable polymer, about 0.175-0.225 g biodegradable polymer, about 0.19-0.21 g biodegradable polymer, about 0.198-0.202 g biodegradable polymer, or about 0.2 g biodegradable polymer per 1 mL solvent. In other words, 20 g biodegradable polymer in 100 mL of solvent would be a biodegradable polymer and solvent solution of 0.2 g biodegradable polymer per 1 mL solvent.

In some embodiments, after the second biodegradable polymer solution is formed, the solution can be added to a mold and/or substrate and created the same way an API layer is made in the mold and/or substrate (drying, removal, etc). In some embodiments, the substrate can be any of the substrates previously described with respect to the API layers. In some embodiments, the mold can be any of the molds previously described with respect to the API layers. In some embodiments, the mold and/or substrate can dictate the shape of the final non-API layer. In other embodiments, the mold and/or substrate forms a precursor non-API layer which can be later modified. In some embodiments, after the second biodegradable polymer solution is formed, the solution can be added to a mold and/or substrate containing an API layer(s) (or another non-API layer(s)). As such, the second biodegradable polymer solution can be added over the API layer (or another non-API layer) in the mold and/or substrate such as a covering or coating over the API layer (or another non-API layer).

In some embodiments, the amount added to the mold and/or substrate can depend on the desired thickness (and degradation rate) of the non-API layer. In some embodiments, about 1-20 mL, about 1-10 mL, about 4-6 mL, or about 5 mL of the biodegradable polymer second solution can be added to the mold. In some embodiments, the biodegradable polymer solution can be added to the substrate and the film applicator and/or spiral bar coater can dictate the thickness of the wet biodegradable polymer solution. In some embodiments, the thickness of the wet layer of biodegradable polymer solution on the substrate can be at least about 0.1 mm, at least about 0.25 mm, at least about 0.5 mm, at least about 0.75 mm, at least about 1 mm, at least about 1.25 mm, at least about 1.5 mm, at least about 1.75 mm, or at least about 2 mm thick. In some embodiments, the thickness of the wet layer of biodegradable polymer solution on the substrate can be at most about 5 mm, at most about 4 mm, at most about 3 mm, at most about 2 mm, at most about 1.5 mm thick, or at most about 1 mm thick.

In some embodiments, the non-API biodegradable solution can be added to the mold or and/or substrate first to form the non-API layer (in a similar manner to the API layer as explained above with respect to the API layer) and then an API layer can be formed on top of the non-API layer in the mold and/or substrate. In some embodiments, the non-API biodegradable solution can be added to the mold and/or substrate first to form the non-API layer (e.g., core or core layer) and then an API layer can be formed on top of the non-API layer in the mold and/or substrate or a different mold so as to surround at least a portion of the non-API layer.

Once on the substrate or in the mold, second biodegradable polymer solution can be dried. In some embodiments, this drying can cause solvent to evaporate, thereby leaving a solidified layer that includes biodegradable polymer and some remaining solvent. In some embodiments, this drying can cause solvent to evaporate, thereby leaving a non-API layer. In some embodiments, this drying can cause solvent to evaporate, thereby leaving a second solidified layer on a side of the first solidified API or non-API layer. In some embodiments, the second layer can be adhered to the first layer via a solvent welding process. In other words, the second layer can adhere to the first layer because the first layer may dissolve slightly when the second layer is applied and can redry with the second layer to form a solid second layer on a side of the solid first layer.

In some embodiments, a non-API layer can also include a small amount of solvent, which will be discussed in further detail below. In some embodiments, the non-API biodegradable polymer solution can be dried in air, nitrogen, or other gases at at least room temperature (e.g., 20-25° C.). In some embodiments, the non-API biodegradable polymer solution can be dried at at least about 20° C., at least about 25° C., or at least about 30° C. In some embodiments, the non-API biodegradable polymer solution can be dried in an environment with a humidity of less than about 30%, less than about 25%, less than about 20%, less than about 15%, less than about 10%, less than 5%, or less than about 1%. In some embodiments, the drying can be for at least about 1 minute, at least about 5 mins, at least about 10 mins, or at least about 15 mins. In some embodiments, the drying can be for at least about 1 minute, at least about 5 mins, at least about 10 mins, at least about 15 mins, at least about 30 mins, at least about 1 hr, at least about 12 hours, or at least about 1 day. In some embodiments, the drying can be at most 6 days, at most 5 days, at most about 2 days, at most about 1 day, at most about 12 hours, at most about 6 hours, at most about 1 hour, or at most about 30 mins. In some embodiments, the layers (API layers, non-API layers, and/or both API and non-API layers) of the drug delivery device can have uniform consistency and be homogeneous.

In some embodiments, after the individual layers (API, non-API) have been added and dried, the layers can be removed. In some embodiments, after the desired amount of API and/or non-API layers have been added and dried together, the API layer, non-API layer, and/or drug delivery film can be removed from the mold or substrate. In some embodiments, the API layer(s) and non-API layer(s) can remain in the mold or on the substrate for further processing (e.g., drying, lamination, cutting, etc.).

In some embodiments, the above process of preparing an API layer and a non-API layer can be repeated for as many additional layers are required for the drug delivery device. In some embodiments, the drug delivery devices can include a plurality of layers instead of one big layer due to manufacturing constraints for one large layer. For example, the thicker an individual layer, the more likely the layer is to have impurities such as gas bubbles which can impact degradation rate. In addition, there is a limit to the thickness of a layer for the solvent to evaporate out to form such a layer. Thus, in some embodiments, the drug delivery devices can be formed from a plurality of layers instead of one layer.

In some embodiments, the desired API layer(s) and/or non-API layer(s) can be laminated together to form a laminated sheet. For example, if an API/API/API layered drug delivery device is required, three individual API layers (having the same and/or different compositions) can be laminated together in the desired order. For example, FIGS. 1A-1C could have been made by laminating together layers 101, 102, 103, 104, and 105 in order. In some embodiments, the laminated sheet is the drug delivery device or a body of the drug delivery device. In some embodiments, the lamination process is a single step. In some embodiments, each layer can be laminated one at a time until the final lamination sheet is created. In some embodiments, a standard laminator can be used for the lamination process such as a laboratory laminator or an industrial laminator. In some embodiments, the laminator may include rolls (e.g., nip rolls) including heated rolls.

In some embodiments, the speed, temperature, pressure, and/or gap parameters of the laminator can be set to the desired parameters. In some embodiments, the speed can be set to at least about 0.5 ft/min, at least about 1 ft/min, at least about 3 ft/min, at least about 5 ft/min, at least about 10 ft/min, or at least about 15 ft/min. In some embodiments, the speed can be set to at most about 20 ft/min, at most about 15 ft/min, at most about 10 ft/min, or at most about 5 ft/min. In some embodiments, the temperature can be set to at least about 50° C., at least about ° C., at least about 100° C., at least about 110° C., at least about 125° C., at least about 150° C., or at least about 200° C. In some embodiments, the temperature can be set to at most about 500° C., at most about 400° C., at most about 300° C., at most about 200° C., at most about 175° C., at most about 150° C., or at most about 125° C. In some embodiments, the pressure can be set to at least about 1 psi, at least about 5 psi, at least about 10 psi, at least about 15 psi, at least about 20 psi, at least about 30 psi, at least about 40 psi, at least about 50 psi, or at least about 75 psi. In some embodiments, the pressure can be set to at most about 200 psi, at most about 150 psi, at most about 100 psi, at most about 75 psi, at most about 50 psi, at most about 40 psi, or at most about 30 psi. In some embodiments, the roller gap of the laminator can be at most about 1 inch, at most about 0.5 inches, at most about 0.25 inches, at most about 0.1 inches, at most about 0.075 inches, or at most about 0.05 inches. In some embodiments, the roller(s) may not have a gap (e.g., nip rolls totally closed).

In some embodiments, for the lamination process, at least two layers (e.g., API/API, non-API/non-API, API/non-API) can be pressed together to form a sandwich. In some embodiments, the at least two layers can be pressed together such that the substrates of the two layers are on the outside (i.e., will contact the rolls of the laminator). In some embodiments, the substrates (e.g., release liners) of the layers contact the rollers of the laminator. In some embodiments, the layer sandwich can be fed to the laminator (e.g., to the rollers of the laminator) to form the laminated sheet.

Once out of the laminator, other layers can be added to the laminated sheet. In some embodiments, depending on the overall layer structure desired for the drug delivery device, a substrate can be removed from one side of the laminated sheet and the process repeated. For example, if two layers were laminated together, a substrate (e.g., release liner) can be removed from one side of the laminated sheet and an additional layer (e.g., API, non-API layer) can be pressed together with the non-substrate side of the laminated sheet to form another sandwich. In some embodiments, this new additional layer can be pressed together such that the substrate of the new layer is on the outside (i.e., will contact the rolls of the laminator). In some embodiments, the substrate (e.g., release liner) of the new layer and the substrate of the old laminated sheet can contact the rollers of the laminator. This new layer sandwich can then be fed to the laminator to form a new laminated sheet. The process can be repeated for as many additional layers as desired in the desired layer structure/orientation/thickness. In some embodiments, once all the layers have been added, the remaining substrates can be removed from the laminated sheet.

Any combination and/or any order of API and/or non-API layers can be created. In some embodiments, any combination and/or any order of API and/or non-API layers can be laminated together. In some embodiments, the drug delivery devices disclosed herein can include any number of API layers and/or non-API layers in any order such as API/API, API/API/API, API/API/API/API, API/API/API/API/API, API/API/non-API/API/API, non-API/non API/non-API, non-API/API, API/non-API/API, non-API/API/non-API, API/API/non-API, non-API/API/API, API/non-API/API/non-API, API/API/non-API/non-API, API/API/API/non-API, non-API/API/non-API/API, non-API/API/API/non-API configurations and so on. These API layers and/or non-API layers can have a composition of any API layer or non-API layer disclosed herein and be made by the same process as any API layer or non-API layer disclosed herein.

In some embodiments, the laminated sheet can be cut into a desired size and/or shape for the drug delivery device. In some embodiments, the laminated sheet can be cut with the substrate or substrates still on the laminated sheet. In some embodiments, the drug delivery device can be cut to be circular, square, rectangular, oval, triangular, diamond shaped, polygon shaped (e.g., pentagon, hexagon, octagon, etc.), arced, trapezoidal, star shaped, or a variety of other shapes and sizes. In some embodiments, the drug delivery device can be cut to be tubular, cylindrical, cone shaped, pyramidal, triangular prism shaped, cube shaped, spherical, rectangular prism-shaped, or a variety of other shapes and sizes. In some embodiments, the laminated sheet can be cut by die cutting, hand cutting, laser cutting, hand punching, waterjet cutting, or similar types of cutting the laminated sheet to form the drug delivery device or body of the drug delivery device. For example, the drug delivery device or body of the drug delivery device can be punched out of the laminated sheet using a die. In some embodiments, the drug delivery film formed by multiple layers of solvent casting in the mold and/or substrate can be cut into a desired size and/or shape for the drug delivery device (i.e., if lamination was not employed). In some embodiments, a layer or layers can be cut into a desired size and/or shape before lamination. In some embodiments, lamination can occur after layers have been cut into a desired size and/or shape.

In some embodiments, the process of preparing an API layer(s) and/or a non-API layer(s) adhered to one another can be repeated for additional layers as well as other API layers and non-API layers adhered to a side of the API layer, other API layers, the non-API layer, and/or other non-API layers. Any combination (see example combinations above) and/or any order of API and/or non-API layers can be obtained by the solvent casting method. In addition, other components of the drug delivery device such as the tip can be created similar to any layer creation discussed herein.

In some embodiments, the drug delivery device can include more than one API layer. In some embodiments, the drug delivery device can include a non-API layer sandwiched between two API layers. In some embodiments, the drug delivery device can include two API layers on top of one another and then a non-API layer on a side of one of the API layers. For example, in some embodiments, the drug delivery device can include a second API layer on a side of the first API layer and a non-API layer on a side of the second API layer opposite the first API layer. In some embodiments, the second API layer can have the same composition (i.e., API) as the first API layer. In some embodiments, a first API layer can degrade faster, slower, or the same as a second API layer. In some embodiments, a first API layer can degrade faster, slower, or the same as a non-API layer. In some embodiments, there may be more than two API layers before a non-API layer. In some embodiments, the drug delivery device may not include a non-API layer. In some embodiments, an API layer(s) can have the same or different composition or the same or different API as another API layer(s). Furthermore, these additional layers can be added to the drug delivery device by the same methods disclosed herein.

In some embodiments, the drug delivery device can include more than one non-API layer. In some embodiments, the drug delivery device can include an API layer sandwiched between two non-API layers. For example, in some embodiments, the drug delivery device can include a first non-API layer on a side of the API layer and a second non-API layer on a side of the API layer opposite the first non-API layer. In some embodiments, the second non-API layer can have the same composition as the first non-API layer. In some embodiments, the second non-API layer can have a different composition as the first non-API layer. In some embodiments, the second non-API layer can degrade faster, slower, or the same as the API layer. In some embodiments, the first non-API layer can degrade faster, slower, or the same as the API layer. In some embodiments, first non-API layer can degrade faster, slower, or the same as the second non-API layer. In some embodiments, there may be more than two non-API layers before an API layer. In some embodiments, a non-API layer(s) can have the same or different composition as another non-API layer(s). Furthermore, these additional layers can be added to the drug delivery device by the same methods disclosed herein.

### Oven Drying

Because the drug delivery devices disclosed herein can be placed directly onto and/or into and/or adjacent to target tissue areas using minimally invasive standard surgical techniques such as open, laparoscopic, endoscopic, percutaneous, or robotic surgery, the drug delivery device should be flexible enough to be used with standard surgical equipment (e.g., a trocar used in laparoscopic surgery, a catheter in endoscopic surgery, a bronchoscope, robotic bronchoscope, a needle for percutaneous delivery, etc.). In some embodiments, the drug delivery devices can be inserted through a 3 mm to 12 mm (e.g., 3, 5, 8, 10, 12 mm trocar) and larger trocar.

In some embodiments, the drug delivery devices can be inserted into a working channel of an endoscope or robotic endoscope such as a bronchoscope or robotic bronchoscope. Once inside the patient, the drug delivery device can be implanted into and/or adjacent to the target tissue site (e.g., tumor) using standard or robotic surgical equipment. In some embodiments, the drug delivery device is placed or implanted in the target tissue such that it resides either completely or partially inside the targeted tissue (i.e., fully or partially embedded inside the targeted tissue).

Solvent can be important because it allows the layer(s) (and overall device) to retain flexibility and not crack during subsequent processing (e.g., lamination, cutting, implantation, etc.). In some embodiments, too much solvent may cause the layer(s) or device to stick/adhere to themselves or itself or other devices such as delivery tools (e.g., endoscope) at warm temperatures. In some embodiments, after removal from the mold and/or substrate, or in some embodiments while still in the mold and/or on the substrate, a layer(s) (e.g., an API layer, a non-API layer) can be placed in an oven for additional drying. In some embodiments, after removal of the drug delivery film (e.g., multiple layers made from solvent casting one on top of each other) from the mold, or in some embodiments while still in the mold, the drug delivery film can be placed in an oven for additional drying. In some embodiments, layers laminated together can be placed in an oven for additional drying. In some embodiments, layers laminated together and/or cut to the shape of the drug delivery device can be placed in an oven for additional drying. In some embodiments, this additional oven drying can cure the polymers in the layers. In some embodiments, this curing can prevent drug separation in an API layer.

In some embodiments, a layer, multiple layers, a drug delivery film, a laminated sheet, or a drug delivery device is placed in an oven at at least about 30° C., at least about 35° C., at least about 36° C., at least about 37° C., at least about 38° C., at least about 39° C., at least about 40° C., at least about 45° C., at least about 50° C., at least about 55° C., at least about 60° C., at least about 65° C., at least about 70° C., at least about 75° C. In some embodiments, a layer, multiple layers, a drug delivery film, a laminated sheet, or a drug delivery device is placed in an oven at at most about 100° C., at most about 90° C., at most about 85° C., at most about 80° C., at most about 75° C., at most about 70° C., at most about 65° C., at most about 60° C., at most about 55° C., at most about at most about 45° C., at most about 42° C., at most about 40° C., at most about 39° C., at most about 38° C., at most about 37° C., at most about 36° C., or at most about 30° C. In some embodiments, a layer, multiple layers, a drug delivery film, a laminated sheet, or a drug delivery device is placed in an oven for at least about 30 mins, at least about 1 hr, at least about 4 hours, at least about 5 hours, at least about 8 hours, at least about 9 hours, at least about 12 hours, at least about 1 day, or at least about 2 days. In some embodiments, a layer, multiple layers, a drug delivery film, a laminated sheet, or a drug delivery device is placed in an oven for at most about days, at most about 4 days, at most about 3 days, at most about 2 days, at most about 1 day, at most about 12 hours, at most about 10 hours, at most about 9 hours, at most about 5 hours, at most about 2 hours, or at most about 1 hour.

Thus, a layer, multiple layers, a drug delivery film, a laminated sheet, or a drug delivery device can have a solvent content of less than about 12 wt. % or about 1-15 wt. %, about 2-12 wt %, about 3-11 wt. %, or about 5-8 wt. %. In some embodiments, an API-layer of the drug delivery device can have at least about 1 wt. %, at least about 2 wt. %, at least about 3 wt. %, at least about 3.5 wt. %, at least about 5 wt. %, at least about 6 wt. %, at least about 7 wt. %, at least about 8 wt. %, or at least about 10 wt. % solvent. In some embodiments, an API layer of the drug delivery device can have at most about 15 wt. %, at most about 12 wt. %, at most about 10 wt. %, at most about 8 wt. %, at most about 7 wt. %, at most about 6 wt. %, or at most about 5 wt. % solvent. In some embodiments, an API layer of the drug delivery device can have about 1-15 wt. %, about 2-12 wt %, about 3-11 wt. %, about 3.5-10 wt. %, or about 5-8 wt. % solvent. In some embodiments, a non-API layer of the drug delivery device can have at least about 1 wt. %, at least about 2 wt. %, at least about 3 wt. %, at least about 3.5 wt. %, at least about 5 wt. %, at least about 6 wt. %, at least about 7 wt. %, at least about 8 wt. %, or at least about 10 wt. % solvent. In some embodiments, a non-API layer of the drug delivery device can have at most about 15 wt. %, at most about 12 wt. %, at most about 11 wt. %, at most about 10 wt. %, at most about 8 wt. %, at most about 7 wt. %, at most about 6 wt. %, or at most about 5 wt. % solvent. In some embodiments, a non-API layer of the drug delivery device can have about 1-15 wt. %, about 2-12 wt %, about 3-11 wt. %, about 3.5-11 wt. %, or about 5-8 wt. % solvent. The amount of solvent in the drug delivery device can be measured by gas chromatography.

In some embodiments, the drug delivery device can be sterilized such as by e-beam radiation. In addition, the drug delivery device can be sealed in a pouch such as a Tyvek pouch to be sterilized and stored in a fridge.

### Orientation Indicator

As explained above, in some embodiments, drug delivery device can include anatomical marker(s). In some embodiments, an API layer and/or a non-API layer can include an anatomical marker(s). In some embodiments, the anatomical marker can be adhered to, connected to, or within the drug delivery device. In some embodiments, the anatomical marker(s) can be adhered to, connected to, or within an API layer and/or a non-API layer. In some embodiments, the anatomical marker(s) can be adhered to connected to, or within another component such as the tip of the drug delivery device. The anatomical marker(s) can be used by a physician to identify the drug delivery device in the patient using a wide variety of imaging techniques. The physician can then monitor the progress and/or location of the drug delivery device with respect to the tumor. In some embodiments, the anatomical marker can be a radiopaque marker, x-ray markers, lead markers, or similar marker(s).

### Implantation

In some embodiments, once a target tissue site (e.g., a tumor) is identified and visualized, the drug delivery device can be implanted within or adjacent to the tumor mass using a variety of delivery methods including, but not limited to, hands, grasping instruments, shafts-attached through the working channel of a scope, etc.

Failure to confirm proper implantation could result in release of the API to non-target sites. For example, if the drug delivery device is not fully embedded within the target tissue (e.g., solid tumor), the portion of the device outside of the target tissue may release API to non-target sites, which can be potentially harmful.

### Drug Delivery Device Properties

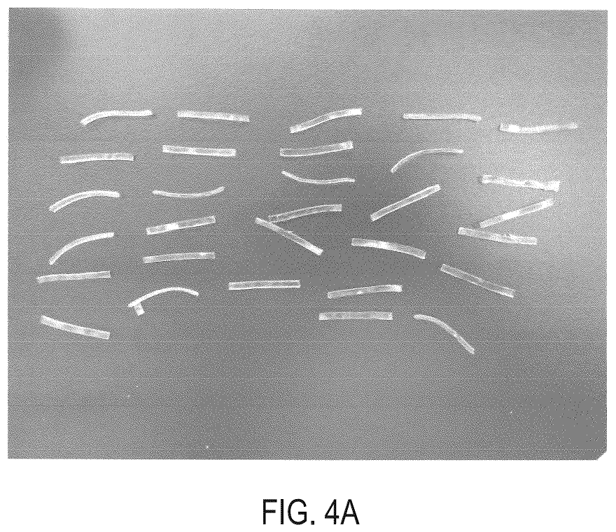

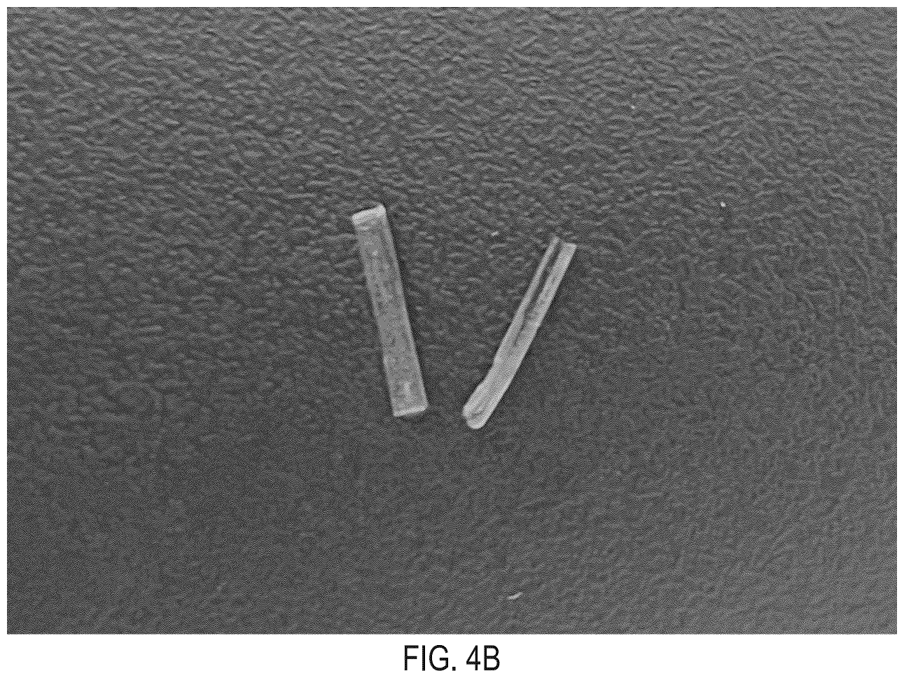

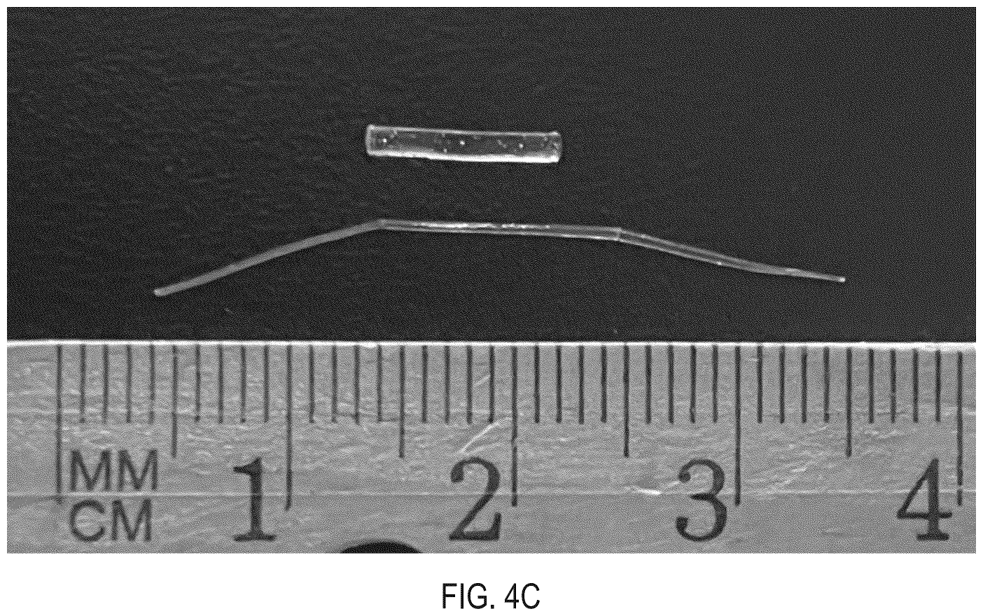

In some embodiments, the drug delivery devices disclose herein can be transparent and/or medium to light brown. The drug delivery devices may have no visible foreign particulate matter on the surface or cracks. FIG. 4A-C illustrates images of drug delivery devices prepared herein with multiple layers laminated together and die cut.

In some embodiments, the average thickness of an API and/or non-API layer can be at least 1 micron, at least 10 microns, at least 25 microns, at least 50 microns, at least 100 microns, at least 250 microns, at least 300 microns, at least 400 microns, at least 500 microns, at least 750 microns, at least 1000 microns, at least 1500 microns, at least 2000 microns, at least 2500 microns, at least 3000 microns, at least 3500 microns, at least 4000 microns, or at least 4500 microns. In some embodiments, the average thickness of an API and/or non-API layer can be at most 5000 microns, at most 4500 microns, at most 4000 microns, at most 3500 microns, at most 3000 microns, at most 2500 microns, at most 2000 microns, at most 1500 microns, at most 1000 microns, at most 750 microns, at most 600 microns, at most 500 microns, at most 400 microns, at most 350 microns, at most 300 microns, at most 250 microns, at most 200 microns, at most 150 microns, at most 100 microns, at most 50 microns, at most 25 microns, or at most 10 microns. In some embodiments, the average thickness of an API and/or non-API layer can be about 50-500 microns, about 100-450 microns, about 150-450 microns, about 200-400 microns, about 215-365 microns, about 250-300 microns, or about 290 microns. In some embodiments, the size of the drug delivery device can be selected based on the desired degradation/API release kinetics. The average thickness can be measured by a micrometer, average of n=5 measurements at 5 randomly selected points on the API and/or non-API layer's surface.

In some embodiments, the width/thickness of the drug delivery device can be at least about 0.1 mm, at least about 0.5 mm, at least about 0.75 mm, at least about 1 mm, at least about 1.25 mm, at least about 1.5 mm, at least about 1.75 mm, at least about 2 mm, at least about 2.5 mm, or at least about 3 mm. In some embodiments, the width/thickness of the drug delivery device can be at most about 10 mm, at most about 5 mm, at most about 4 mm, at most about 3 mm, at most about 2.5 mm, at most about 2 mm, at most about 1.75 mm, at most about 1.5 mm, at most about 1.25 mm, or at most about 1 mm. In some embodiments, the width/thickness of the drug delivery device is about 0.1-10 mm, about 0.5-5 mm, about 0.75-2.5 mm, or about 1-2 mm. In some embodiments, the height of the drug delivery device can be at least about 0.1 mm, at least about 0.5 mm, at least about 0.75 mm, at least about 1 mm, at least about 1.25 mm, at least about 1.5 mm, at least about 1.75 mm, at least about 2 mm, at least about 2.5 mm, or at least about 3 mm. In some embodiments, the height of the drug delivery device can be at most about 10 mm, at most about 5 mm, at most about 4 mm, at most about 3 mm, at most about 2.5 mm, at most about 2 mm, at most about 1.75 mm, at most about 1.5 mm, at most about 1.25 mm, or at most about 1 mm. In some embodiments, the height of the drug delivery device is about 0.1-10 mm, about 0.5-5 mm, about 0.75-2.5 mm, or about 1-2 mm. In some embodiments, the width, thickness, and/or height of the drug delivery device can be measured by a micrometer.

In some embodiments, the length of the drug delivery device can be at least about 0.1 cm, at least about 0.5 cm, at least about 0.75 cm, at least about 1 cm, at least about 1.25 cm, at least about 1.5 cm, at least about 1.75 cm, at least about 2 cm, at least about 2.5 cm, or at least about 3 cm. In some embodiments, the length of the drug delivery device can be at most about 10 cm, at most about 5 cm, at most about 4 cm, at most about 3 cm, at most about 2.5 cm, at most about 2 cm, at most about 1.75 cm, at most about 1.5 cm, at most about 1.25 cm, or at most about 1 cm. In some embodiments, the length of the drug delivery device is about 0.1-10 cm, about 0.5-5 cm, about 0.75-2.5 cm, or about 1-2 cm. In some embodiments, the length of the drug delivery device can be measured using a ruler or similar measuring tool.

In some embodiments, the drug delivery device can be configured to release the API according to a defined release kinetic profile. In some embodiments, the release of the API can be delayed (or only a sub-therapeutically effective amount of the API can be released during the delay period) after the device is implanted at and/or in the target tissue site such that the patient's body can recover from the implantation surgery prior to releasing the drug. The delay can allow for some healing at the implantation site before the release of the API, thereby potentially reducing risks associated with swelling, perforation, bleeding, infection, and other potential issues. For example, in some embodiments, the drug delivery device may have a layer and/or coating of a non-API composition on all sides of the drug delivery device such that no API will be released while the patient heals from implantation surgery. Instead, only this layer and/or coating of the non-API coating can degrade. In some embodiments, the API delay release period can be at least 1 day, at least 3 days, at least 7 days, at least 9 days, at least 10 days, at least 12 days, at least 14 days, at least 16 days, at least 18 days, or at least 21 days. In some embodiments, the API delay release period can be at most 28 days, at most 25 days, at most 21 days, at most 18 days, at most 15 days, at most 14 days, at most 12 days, at most 10 days, at most 8 days, at most 7 days, at most 5 days, or at most 3 days. In some embodiments, the API delay release period can be 1-28 days, 1-21 days, 1-14 days, or 7-14 days. After the delay period, the API can have a substantially linear or linear release rate.

In some embodiments, an API layer with PLGA 50:50 can begin to release the API at the target tissue at approximately 1 week after implantation and the API can be fully released by 4 weeks after implantation. This can align well with the degradation data—it takes time for the polymer to hydrate, but at 4 weeks most of the PLGA 50:50 is gone, so most of the API will have been released. In addition, a non-API layer made with PLGA 75:25 can serve as a mechanism that maintains structure, strength, and/or support for the drug delivery device as it can degrade slower. This can help anchor the device to the target site.

The degradation of biodegradable polymer in an API layer can control the release the API from the API layer during use. As such, degradation of an API layer in the drug delivery device can be tuned based on the biodegradable polymer in the API layer as well as the thickness of the API layer as the thicker the API layer the longer it will take to degrade. In some embodiments, an API layer can be configured to completely degrade within a period of at least 3 days, at least 5 days, at least 1 week, at least 2 weeks, at least 3 weeks, at least 3.5 weeks, at least 4 weeks, at least 30 days, at least 4.5 weeks, at least 5 weeks, at least 6 weeks, at least 7 weeks, at least 8 weeks, at least 9 weeks, at least 10 weeks, at least 2 months, at least 3 months, at least 4 months, at least 6 months, at least 8 months, or at least 10 months, after implantation. In some embodiments, an API layer can be configured to completely degrade within a period of at most 2 years, at most 1 year, at most 10 months, at most 8 months, at most 6 months, at most 4 months, at most 3 months, at most 2 months, at most 10 weeks, at most 9 weeks, at most 8 weeks, at most 6 weeks, at most 5 weeks, at most 4.5 weeks, at most 30 days, at most 4 weeks, at most 3.5 weeks, at most 3 weeks, at most 2 weeks, or at most 1 week, after implantation. In some embodiments, an API layer can be configured to completely degrade within a period of about 3 days to 10 months, 1 week to 6 months, 1-6 weeks, about 2-6 weeks, about 3-5 weeks, about 3.5-4.5 weeks, or about 4 weeks (about 30 days), after implantation. “Completely degrade” or “fully degrade” used herein refers to a layer or device degrading to less than 10% of original mass within the time period. In some embodiments, the API can be released from an API layer at a rate of at least about 0.01 mg/day, at least about 0.05 mg/day, at least about 0.1 mg/day, at least about 0.5 mg/day, at least about 0.75 mg/day, or at least about 1 mg API/day. In some embodiments, the API can be released from an API layer at a rate of at least about 0.1 mg/week, at least about 0.5 mg/week, at least about 0.75 mg/week, or at least about 1 mg API/week. In some embodiments, the API can be released from the API layer at a rate of about 0.01-10 mg/day, about 0.1-10 mg/day, about 0.1-5 mg/day, about 0.1-3 mg/day, about 0.1-1 mg/day, about 1-5 mg/day, about 2-5 mg/day, or about 3-4 mg/day, after implantation. In some embodiments, the API can be released from an API layer at a rate of about 0.5-70 mg/week, about 0.5-35 mg/week, about 0.5-20 mg/week, about 0.5-10 mg/week, about 0.5-5 mg/week, about 0.5-1 mg/week, about 7-70 mg/week, about 7-35 mg/week, about 14-35 mg/week, or about 21-28 mg/week, after implantation. In some embodiments, the degradation profile of an API layer (and the API release profile) after implantation can include a delay period from about 1 day to 2 weeks. After the delay period, the degradation of an API layer (and the API release) can be substantially linear or linear. In some embodiments, the outer layers (e.g., layers 101 and 105 of FIGS. 1A-1C) may degrade at a faster rate than inner layers (e.g., layers 102, 103, 104) of the drug delivery device because more surface area of these layers is exposed to the degrading environment.

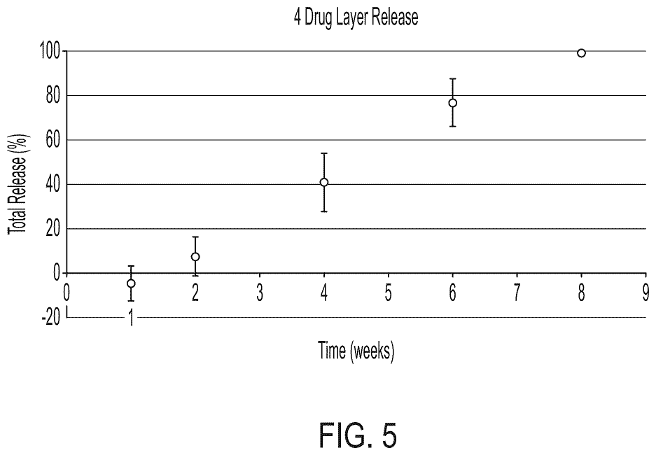

Drug release (i.e., API release) was tested in vitro for the drug delivery devices disclosed herein. Specifically, drug delivery devices containing four layers of about 0.7 g of PLGA 50:50, 100 mg paclitaxel, and 4 mg acetone were created and tested for drug release rates. All four layers were created individually and then the drug delivery devices were laminated together one layer at a time. The laminated sheet was die cut to form a drug delivery device of about 1.5 mm thick, about 1.25 mm wide, and about 2 cm long. Drug delivery devices were made such that 6 products at each timepoint (1, 2, 3, 4 and 6 weeks) were available for testing. Each sample was placed in a scintillation vial with 20 ml of 1.75M Sodium Salicylate (release solution). Samples were held in a water bath at 37C until their timepoint. Once pulled, the release solution was discarded and the sample was fully dissolved in Acetonitrile. An assay for paclitaxel was run on the dissolved sample on HPLC to determine the remaining drug content, which was used to determine the quantity of drug released. Samples were taken at weeks 1, 2, 4, 6, and 8. FIG. 5 illustrates the total percent release of the drug from the drug delivery device over time. Results showed a 1-2 week delay of release of API, followed by steady linear release up until week 8.

In addition, degradation of a non-API layer in the drug delivery device can be tuned based on the biodegradable polymer in the non-API layer as well as the thickness of the non-API layer. In some embodiments, the non-API layer can be configured to degrade at a slower rate than an API layer such that the API is released toward the targeted tissue. In some embodiments, the non-API layer can be configured to degrade at the same rate as an API layer. In some embodiments, the non-API layer can be configured to degrade at a faster rate as an API layer. For example, in some embodiments, there may be a non-API layer or non-API coating that covers all sides of the drug delivery device. This non-API layer/coating can degrade faster than the API layer such that there may be a delayed period for API release due to the degradation of the non-API layer/coating first.

In some embodiments, a non-API layer can be configured to completely degrade within a period of at least 5 days, at least 1 week, at least 2 weeks, at least 3 weeks, at least 4 weeks, at least 5 weeks, at least 6 weeks, at least 8 weeks, at least 10 weeks, at least 11 weeks, at least 12 weeks, at least 4 months, at least 5 months, at least 6 months, at least 8 months, at least 10 months, or at least 1 year, after implantation. In some embodiments, a non-API layer can be configured to completely degrade within a period of at most 3 years, at most 2 years, at most 1 year, at most 10 months, at most 8 months, at most 6 months, at most 4 months, at most 3 months, at most 12 weeks, at most 11 weeks, at most 10 weeks, at most 8 weeks, at most 6 weeks, at most 5 weeks, at most 4 weeks, at most 3 weeks, or at most 2 weeks, after implantation. In some embodiments, a non-API layer can be configured to completely degrade within a period of about 1 week to 2 years, 1 week to 1 year, 1 week to 6 months, about 4-14 weeks, about 6-12 weeks, about 8-12 weeks, about 9-11 weeks, or about 10 weeks, after implantation.

### Cancer Treatment

In some embodiments, the targeted tissue may be tissue associated with various organs throughout the body including, but not limited to, pancreas, biliary system, gallbladder, esophageal system, liver, stomach, peritoneum, small bowel, lung, colon, or metastasis from a primary tumor. For example, the targeted tissue may be cancerous tissue/cells (e.g., a tumor or tumors) on the pancreas, biliary system, gallbladder, esophageal system, liver, stomach, peritoneum, small bowel, lung, colon, or metastasis from a primary tumor. As such, the drug delivery devices disclosed herein can be used for treating many diseases including tumors of various organs throughout the body including the pancreas, biliary system, gallbladder, esophageal system, liver, stomach, peritoneum, small bowel, lung, colon, or metastasis from a primary tumor. Specifically, the various cancers that the drug delivery device can help treat include, but are not limited to, pancreatic ductal adenocarcinoma (PDAC), cholangiocarcinoma, gallbladder cancer, lymphoma, non-small cell lung cancer, or metastatic tumors. In some embodiments, the drug delivery devices can be used to treat resectable cancers and/or non-resectable cancers. In some embodiments, the drug delivery devices herein can be used to treat non-immediately resectable to non-metastatic cancers. In some embodiments, the drug delivery devices can be used to treat patients after cancers are resected to prevent recurrences. For example, the drug delivery devices can be used for treatment of patients with borderline resectable or locally advanced pancreatic adenocarcinoma or lung cancers (e.g., non-small cell lung cancer). In some embodiments, the cancer treated by the drug delivery device can be a tumor in the tissue of primary origin or metastatic spread to the tissue. In some embodiments, the drug delivery devices can be used for treatment of patients with non-immediately resectable cancer, non-metastatic cancer, borderline resectable cancer, resectable cancer, locally advanced cancer, metastatic cancer, and/or metastasis from a primary tumor.

In some embodiments, the drug delivery devices disclosed herein can be placed into and/or adjacent to a tumor of interest (i.e., intratumorally) and can biodegrade within the body of the patient in about 1 week to 2 years, about 1-52 weeks, about 1-26 weeks, about 1-24 weeks, about 1-20 weeks, about 1-15 weeks, about 4-12 weeks, about 6-12 weeks, about 8-12 weeks, about 9-11 weeks, or about 10 weeks of implantation. In some embodiments, the tumor may be on the inside of the organ (i.e., not on the surface) and the drug delivery device is placed within the tumor. As explained above, the drug delivery device can be placed directly into and/or adjacent to a tumor using minimally invasive standard surgical techniques during routinely performed staging evaluations. In some embodiments, multiple drug delivery devices can be placed directly into and/or adjacent to a targeted tissue (e.g., simultaneously and/or in series after one has finished degrading). In some embodiments, the drug delivery device can be inserted into a working channel of an endoscope, guided to the tumor of interest, and inserted into and/or adjacent to the tumor of interest.

In some embodiments, the degradation of an API layer can control the release of the API for a period of at least 3 days, at least 5 days, at least 1 week, at least 2 weeks, at least 3 weeks, at least 3.5 weeks, at least 4 weeks, at least 30 days, at least 4.5 weeks, at least 5 weeks, at least 6 weeks, at least 2 months, at least 3 months, at least 4 months, at least 6 months, at least 8 months, or at least 10 months, after implantation. In some embodiments, the degradation of an API layer can control the release of the API for a period of at most 2 years, at most 1 year, at most 10 months, at most 8 months, at most 6 months, at most 4 months, at most 3 months, at most 2 months, at most 6 weeks, at most 5 weeks, at most 4.5 weeks, at most 30 days, at most 4 weeks, at most 3.5 weeks, at most 3 weeks, at most 2 weeks, or at most 1 week, after implantation. In some embodiments, the degradation of an API layer can control the release of the API for a period of about 3 days to 10 months, 1 week to 6 months, 1-6 weeks, about 2-6 weeks, about 3-5 weeks, about 3.5-4.5 weeks, or about 4 weeks (about 30 days), after implantation. In some embodiments, the biodegradable polymer in an API layer can provide controlled and sustained release of the API over this period during which the cancer-facing side of the device is absorbed into the body. In some embodiments, a non-API layer(s) of the device and/or tip, can help ensure that the drug delivery device maintains contact with the targeted tissue (e.g., tumor) during drug release and can help prevent the drug delivery device from leaving or releasing from the area of interest. This non-API layer(s) and/or tip can then completely degrade after the API layer has completely degraded and released the entirety of the API. In some embodiments, a non-API layer can completely degrade at the same time or faster than the API layer.

In some embodiments, the drug delivery devices disclosed herein can stabilize and/or reduce the size of a tumor after implantation on a tumor. In some embodiments, the reduction in size of the tumor, the reduction in tumor volume, the reduction in the largest dimension of the tumor, and/or the reduction of the anterior/posterior diameter orthogonal to the drug delivery device can be after the drug delivery device has completely dissolved in the patient, after the drug delivery device has partially dissolved in the patient, and/or after a portion of the drug delivery device (e.g., an API layer) has completely dissolved in the patient.

In some embodiments, the drug delivery devices and methods disclosed herein can be used together with systemic chemotherapy, radiation therapy, and/or surgery. In some embodiments, the drug delivery devices and methods disclosed herein can improve tumor penetration of systemic chemotherapy in a patient. In some embodiments, systemic chemotherapy can be administered (or start being administered) after implantation (e.g., two weeks, 3 weeks, 4 weeks, etc. after implantation) of the drug delivery device. In some embodiments, systemic chemotherapy can be administered (or start being administered) at least 1 week, at least 2 weeks, at least 3 weeks, at least 4 weeks, at least 3 months, at least 6 months, or at least one year after implantation of the drug delivery device.

The drug delivery devices disclosed herein can change the route of administration to target just the area of interest, thereby increasing the amount of drug reaching the tumor with the aim to enhance therapeutic efficacy. As such, the drug delivery device can be applied in patients with lung cancer (among other cancers): (i) pre-operatively as neoadjuvant treatment to control progression and downsize to improve resectability and/or clinical benefit (i.e. easiness of breath, reduction in pain); (ii) post-resection to reduce the rate of local recurrence; or (iii) in metastatic patient to control local progression and improve quality of life.

### Additional Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Reference to “about” a value or parameter herein includes (and describes) variations that are directed to that value or parameter per se. For example, description referring to “about X” includes description of “X”. In addition, reference to phrases “less than”, “greater than”, “at most”, “at least”, “less than or equal to”, “greater than or equal to”, or other similar phrases followed by a string of values or parameters is meant to apply the phrase to each value or parameter in the string of values or parameters. For example, a statement that a layer has a thickness of at least about 5 cm, about 10 cm, or about 15 cm is meant to mean that the layer has a thickness of at least about 5 cm, at least about 10 cm, or at least about 15 cm.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of a pathological consequence of a cancer. The methods of the invention contemplate any one or more of these aspects of treatment.

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

The above description is presented to enable a person skilled in the art to make and use the disclosure, and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

1. A drug delivery device comprising:
a body comprising a plurality of layers, wherein the plurality of layers comprises at least one first layer comprising an active pharmaceutical ingredient (API) and a biodegradable polymer,
wherein the drug delivery device is configured to be inserted into a target tissue site of a patient.

2. The drug delivery device of claim 1, further comprising a tip connected or integrated to a distal end of the body of the drug delivery device.

3. The drug delivery device of claim 2, wherein the drug delivery device is configured to be inserted into a target tissue site of the patient tip first.

4. The drug delivery device of claim 1, wherein the target tissue site is a tumor of the pancreas, biliary system, gallbladder, esophageal system, liver, stomach, peritoneum, small bowel, lung, or colon.

5. The drug delivery device of claim 1, wherein the biodegradable polymer is poly(lactic-co-glycolic acid) (PLGA).

6. The drug delivery device of claim 5, wherein the biodegradable polymer is PLGA 50:50.

7. The drug delivery device of claim 1, wherein the at least one first layer comprises 1-15 wt. % API.

8. The drug delivery device of claim 1, wherein the at least one first layer comprises a solvent.

9. The drug delivery device of claim 8, wherein the at least one first layer comprises 1-15 wt. % solvent.

10. The drug delivery device of claim 9, wherein the solvent is acetone.

11. The drug delivery device of claim 1, wherein the plurality of layers comprises at least one second layer comprising a second biodegradable polymer, wherein the second biodegradable polymer has a slower degradation rate than the first biodegradable polymer.

12. The drug delivery device of claim 11, wherein the second biodegradable polymer is PLGA 75:25.

13. The drug delivery device of claim 1, wherein the plurality of layers comprises at least one third layer comprising a second API and a third biodegradable polymer.

14. The drug delivery device of claim 1, wherein the plurality of layers comprises at least one fourth layer comprising a third API and a fourth biodegradable polymer.

15. The drug delivery device of claim 1, wherein each of the plurality of layers has the same composition.

16. The drug delivery device of claim 1, wherein the drug delivery device is configured to be implanted in a patient using standard open and/or minimally invasive procedures.

17. The drug delivery device of claim 1, wherein the drug delivery device is configured to be inserted into a target tissue site of a patient using a bronchoscope, forceps, trocar, or needle.

18. The drug delivery device of claim 1, wherein the drug delivery device is configured to be inserted into a target tissue site of a patient robotically.

19. The drug delivery device of claim 1, wherein the at least one first layer is configured to release the API when inserted into a target tissue site of a patient.

20. The drug delivery device of claim 19, wherein the at least one first layer is configured to release the API multi-directionally when inserted into the target tissue site of the patient.

21. The drug delivery device of claim 19, wherein the release of the API is controlled by in vivo degradation of the biodegradable polymer in the target tissue site.

22. The drug delivery device of claim 2, wherein the tip comprises a fifth biodegradable polymer that has a slower degradation rate than the first biodegradable polymer.

23. The drug delivery device of claim 22, wherein the tip comprises PLGA 75:25.

24. The drug delivery device of claim 2, wherein the tip comprises a tissue retaining mechanism.

25. The drug delivery device of claim 24, wherein the tissue retaining mechanism is configured to hold the drug delivery device in a target tissue when inserted in the target tissue of a patient.

26. The drug delivery device of claim 25, wherein the tissue retaining mechanism extends outwardly from the tip.

27. The drug delivery device of claim 26, wherein the tissue retaining mechanism comprises barbs and/or edges.

28. The drug delivery device of claim 2, wherein the tip has at least one vertex at its distal end.

29. The drug delivery device of claim 1, wherein the body comprises a disengagement mechanism towards a proximal end of the body.

30. The drug delivery device of claim 29, wherein a flexible shaft is connected to the proximal end of the body.

31. The drug delivery device of claim 30, wherein the disengagement mechanism is configured to disengage the drug delivery device from the flexible shaft.

32. The drug delivery device of claim 30, wherein the disengagement mechanism is configured to disengage the drug delivery device from the flexible shaft via twisting.

33. A method of preparing a drug delivery device comprising:
adding a first solution comprising a first biodegradable polymer, an active pharmaceutical ingredient (API), and a solvent to a first substrate;
drying the first solution on the first substrate to form a first layer;
adding a second solution comprising a second biodegradable polymer and a second solvent to a second substrate;
drying the second solution on the second substrate to form a second layer;
laminating the first layer and the second layer together to form a laminated sheet.

34. The method of claim 33, cutting the laminated sheet to form a body of a drug delivery device.

35. The method of claim 34, wherein the cutting is die cutting.

36. The method of claim 33, wherein the second solution comprises a second API.

37. The method of claim 34, wherein the first API and the second API are the same.

38. The method of claim 33, further comprising:
adding a third solution comprising a third biodegradable polymer and a third solvent a third substrate;
drying the third solution on the third substrate to form a third layer;
laminating the third layer to the laminated sheet to form a second laminated sheet.

39. The method of claim 38, wherein the third solution comprises a third API.

40. The method of claim 33, further comprising:
spreading the first solution across the first substrate with a film applicator; and
spreading the second solution across the second substrate with a film applicator.

41. The method of claim 40, wherein the spread first solution and the spread second solution have a desired thickness pursuant to the film applicator.

42. The method of claim 33, wherein the first and/or second substrate comprises a release liner.

43. The method of claim 33, further comprising heating the first and second layers in an oven after drying the first and second solutions.

44. The method of claim 34, attaching a tip to a distal end of the body of the drug delivery device.

45. A method of treating tissue of a patient, comprising:
implanting a drug delivery device into a target tissue site of a patient, the drug delivery device comprising:
a body comprising a plurality of layers, wherein the plurality of layers comprises at least one first layer comprising an active pharmaceutical ingredient (API) and a biodegradable polymer; and
releasing the API from the at least one first layer in the target tissue site, wherein the release of the API is controlled by in vivo degradation of the biodegradable polymer in the target tissue site.

46. The method of claim 45, wherein the drug delivery device comprises a tip connected to a distal end of the drug delivery device.

47. The method of claim 46, wherein the drug delivery device is implanted tip first into the target tissue site of the patient.

48. The method of claim 45, wherein the target tissue site is a tumor of the pancreas, biliary system, gallbladder, esophageal system, liver, stomach, peritoneum, small bowel, lung, or colon.

49. The method of claim 45, wherein the drug delivery device is implanted via open surgery, laparoscopically, percutaneously, robotically, or endoscopically.

50. The method of claim 49, wherein the drug delivery device is implanted via a bronchoscope.

51. The method of claim 45, wherein releasing the API from the at least one first layer comprises releasing the API multi-directionally away from drug delivery device.

52. The method of claim 46, wherein the drug delivery device is implanted into the target tissue site of the patient such that the tip is configured to hold the drug delivery device in the target tissue.

53. The method of claim 45, wherein implanting the drug delivery device comprises using a tool to deliver the drug delivery device to the target tissue site of the patient.

54. The method of claim 53, wherein the tool comprises a flexible shaft and is connected to a proximal end of the body of the drug delivery device.

55. The method of claim 54, wherein implanting the drug delivery device into the target tissue site comprises disengaging the body of the drug delivery device from the flexible shaft.

56. The method of claim 55, wherein disengaging the drug delivery device from the flexible shaft comprises twisting the flexible shaft with respect to the drug delivery device.

57. The method of claim 45, wherein the body of the drug delivery device comprises an anatomical marker and implanting the drug delivery device into the target tissue site of the patient comprises monitoring a location the anatomical marker.

58-108. (canceled)

109. The method of claim 45, wherein the release of the API follows a delay period after implantation.

110. The method of claim 109, wherein a sub-therapeutically effective amount of the API is released during the delay period.

111. The method of claim 109, wherein after the delay period, the API is released at a substantially linear or linear release rate.

112. The drug delivery device of, wherein the body of the drug delivery device is flexible.

113. The drug delivery device of claim 19, wherein the at least one first layer is configured to release the API unidirectionally when inserted into the target tissue site of the patient.

114. A method of preparing a drug delivery device comprising:
adding a first solution comprising a first biodegradable polymer, an active pharmaceutical ingredient (API), and a solvent to a first substrate;
drying the first solution on the first substrate to form a first layer;
adding a second solution comprising a second biodegradable polymer and a second solvent over the first layer; and
drying the second solution over the first layer to form a second layer on a side of the first layer.

115. The method of claim 114, wherein the second solution comprises a second API.

116. The method of claim 115, wherein the first API and the second API are the same.

117. The method of claim 114, further comprising:
adding a third solution comprising a third biodegradable polymer and a third solvent over the second layer; and
drying the third solution over the second layer to form a third layer on a side of the second layer opposite the first layer.

118. The method of claim 117, wherein the third solution comprises a third API.

119. The method of claim 114, further comprising heating the first and second layers in an oven after drying the first and second solutions.

120. The method of claim 114, attaching a tip to a distal end of the body of the drug delivery device.
